(19) United States
(12) Patent Application Publication
(10) Pub. No.: US 2026/0146235 A1
(43) Pub. Date: May 28, 2026

(54) CELLS AND PREPARATION AND USES THEREOF

(71) Applicant: LifeNet Health, Virginia Beach, VA (US)

(72) Inventors: Kristina Wolf, Virginia Beach, VA (US); Edward LeCluyse, Virginia Beach, VA (US); Jessica Weaver, Virginia Beach, VA (US); Sharon Presnell, Virginia Beach, VA (US); Justin Odanga, Virginia Beach, VA (US); Jingsong Chen, Virginia Beach, VA (US)

(21) Appl. No.: 19/340,050

(22) Filed: Sep. 25, 2025

Related U.S. Application Data
(60) Provisional application No. 63/700,407, filed on Sep. 27, 2024.

(51) Int. Cl. C12N 5/071; C12N 5/00; G01N 33/50

(52) U.S. Cl. CPC C12N 5/067; C12N 5/0081; G01N 33/5067; G01N 2500/10

(57) ABSTRACT

A method for preparing enriched cells is provided. The cells may be from a single donor or from two or more donors. The method includes removing dying or dead cells from primary cells from the donor(s) by a density gradient separation to generate separated cells; cryopreserving the separated cells to generate cryopreserved separated cells; thawing the cryopreserved separated cells to generate thawed separated cells; and removing dying or dead cells from the thawed separated cells by subsequent density gradient separation(s). The primary cells from two or more donors may be combined before or after the first density gradient separation to provide pooled enriched cells. The enriched cells may have a viability rate greater than the primary cells from the donor(s). Also provided is a product including the enriched cells (which may be pooled enriched cells) and methods for using the enriched cells or the product including the enriched cells.

### CROSS-REFERENCE TO RELATED APPLICATION

This application claims priority to U.S. Provisional Patent Application No. 63/700,407, filed Sep. 27, 2024, titled “CELLS AND PREPARATION AND USES THEREOF” the entire contents of which are incorporated herein by reference in its entirety for all purposes.

### FIELD OF THE INVENTION

The invention relates generally to single donor and pooled cells, for example, hepatocytes or thyrocytes or AT2 lung cells or pancreatic islets, and preparation and uses thereof.

### BACKGROUND OF THE INVENTION

Primary human liver cells, such as hepatocytes, isolated from donors have been used in 2D in vitro culture systems for studying drug metabolism and transport or drug induced liver injury. In 2D in vitro culture systems, primary human liver cells are maintained in a culture medium and attach to a surface (e.g., a plate) to generate a monolayer of the liver cells suitable for testing drug metabolism, transport, toxicity or viral infection. For 2D in vitro culture systems, is isolated primary human liver cells having a plateability of at least ≥85% for a prolonged period of time, for example, at least 1, 2, 3, 4, 5, 6, 7, 14, 21, 28, 35, 42 or 49 days, are desirable while isolated primary liver cells having a plateability of less than 70% are generally deemed less desirable and not suited for long-term experiments. A high percentage of cryopreserved primary human liver cell batches fall into the latter category of non- or poorly plateable liver cells, limiting the availability of many batches with desirable donor specifications such as non-alcoholic fatty liver disease (NAFLD) and nonalcoholic steatohepatitis (NASH) for applications such as testing drug metabolism and transport, toxicity or viral infection.

There is a need to improve heterogeneity, viability, functional stability, and culture longevity of pooled cells, for example, pooled liver cells such as hepatocytes, Kupffer cells, stellate cells, sinusoidal endothelial cells; lung cells such as alveolar type 2 (AT2) lung cells; thyrocytes; or pancreatic islets.

There is also a need for increasing the availability of cell batches for desirable phenotypes such as those with non-alcoholic fatty liver disease (NAFLD) and non-alcoholic steatohepatitis (NASH); thyroid disease such as hyperthyroidism, hypothyroidism, Hashimoto's thyroiditis, Graves' disease, goiter, thyroid nodules, thyroid tumors, or thyroid cancer; lung disease such as idiopathic pulmonary fibrosis (IPF) which may include a pro-fibrotic phenotype, lung adenocarcinoma (LUAD), acute lung injury (ALI) (e.g. pneumonia, inhalation of toxic substances, fat embolism, viral infection of the lungs), acute respiratory distress syndrome (ARDS), hyperplasia, or chronic obstructive pulmonary disease (COPD); pancreatic disorders such as pancreatitis, pancreatic cancer, or type 1 or type 2 diabetes mellitus (T1DM or T2DM).

### SUMMARY OF THE INVENTION

The present invention relates to pooled cells, for example, pooled liver cells or thyrocytes or AT2 lung cells or pancreatic islets, and preparation and uses thereof. The invention also relates to methods of preparing and using these cells, such as liver cells, especially for preparing the cells to provide increased heterogeneity, viability and at the same time, providing an increased population of desirable phenotypes such as but not limited to non-alcoholic fatty liver disease (NAFLD), non-alcoholic steatohepatitis (NASH), type 2 diabetes mellitus (T2DM), benign simple steatosis, fibrosis, cirrhosis, and hepatocellular carcinoma (HCC). If the cells are thyrocytes, examples of diseases that are desirable to represent are hyperthyroidism, hypothyroidism, Hashimoto's thyroiditis, Graves' disease, goiter, thyroid nodules, thyroid tumors, or thyroid cancer. If the cells are lung cells, examples of lung diseases that are desirable to represent are idiopathic pulmonary fibrosis (IPF) which may include a pro-fibrotic phenotype, lung adenocarcinoma (LUAD), acute lung injury (ALI) (e.g. pneumonia, inhalation of toxic substances, fat embolism, viral infection of the lungs), acute respiratory distress syndrome (ARDS), hyperplasia, or chronic obstructive pulmonary disease (COPD). If the cells are pancreatic islets, examples of pancreatic disorders that are desirable to represent are pancreatitis, pancreatic cancer, or type 1 or type 2 diabetes mellitus (T1DM or T2DM).

Liver diseases are a global health concern. Many in vitro liver models utilize cryopreserved primary human hepatocytes (PHHs), which commonly undergo post-thaw processing through colloidal silica gradients to remove debris and enrich the viable cell population. Post-thaw processing effects on healthy PHHs is partially understood, but the consequences of applying post-thaw density gradient separation to disease-origin PHHs have not been described. Using the TruVivo® cell system [an all-human triculture system (hTCS)], normal, diseased, and type 2 diabetes mellitus (T2DM) PHHs were cultured for 14 days after initially being subjected to either low-density (permissive) or high-density (selective) gradients using Percoll®-based thawing medium. According to some embodiments “low density” Percoll® is about 6.8% Percoll®. This Percoll® amount is low compared to the amount of Percoll® in the thawing medium typically used, which is about 27% Percoll®. Differences in morphology and attachment were observed in low density Percoll® enriched PHHs. Changes in functionality, including albumin and urea secretion and cytochrome P450 3A4 (CYP3A4) enzyme activity, were measured in diseased, T2DM, and fibrotic PHHs enriched in low Percoll® compared to PHHs enriched in high Percoll®. Lipogenesis was increased in these PHHs enriched in low Percoll®. Higher expression of cytokeratin 18 (CK18) and transforming growth factor-beta (TGF-0), two fibrotic markers, and changes in expression of the macrophage markers cluster of differentiation 68 (CD68) and cluster of differentiation 163 (CD163) were also measured. In conclusion, the use of Percoll® for enrichment of PHHs post-thaw, in addition to the use of Percoll® before cryopreserving the PHHs, results in differences in attachment and functionality along with changes in diseased phenotypes in the TruVivo® cell system.

The present invention also relates to other pooled or single donor epithelial cells, such as thyrocytes, AT2 lung cells or pancreatic islets.

For all these cell types, it is also desirable to enrich the heterogeneity of cells for pooled cell populations. Heterogeneity may be in terms of any combination of age, race, or disease state for example. Heterogeneity may also comprising pooling different cell types. For example, the dermal fibroblasts and endothelial cells used as feeder cells may be pooled. Hepatic stellate cells or sinusoidal endothelial cells (or even macrophages) may be pooled. Other combinations of calls and cell types are envisaged.

According to an embodiment, a method for preparing pooled enriched cells is provided. The method comprises:
(a) obtaining primary cells from two or more donors;
(b) combining the human primary cells from the two or more donors, whereby combined primary cells are generated;
(c) removing dying or dead cells from the combined human primary cells by a first density gradient separation, whereby combined separated cells are generated;
(d) cryopreserving the combined separated cells, whereby cryopreserved combined separated cells are generated;
(e) thawing the cryopreserved combined separated cells, whereby thawed combined separated cells are generated; and
(f) removing dying or dead cells from the thawed combined separated cells by a second density gradient separation, whereby pooled enriched cells are prepared, wherein the pooled enriched cells have a viability rate greater than that of the primary cells from at least one of the two or more donors.

According to another embodiment, another method for preparing pooled enriched cells is provided. This method comprises:
(a) obtaining primary cells from two or more donors;
(b) removing dying or dead cells from the primary cells from each of the two or more donors by a first density gradient separation, whereby separated cells from each of the two or more donors are generated;
(c) combining the separated cells from the two or more donors, whereby combined separated cells are generated;
(d) cryopreserving the combined separated cells, cryopreserved combined separated cells are generated;
(e) thawing the cryopreserved combined separated cells, whereby thawed combined separated cells are generated; and
(f) removing dying or dead cells from the thawed combined separated cells by a second density gradient separation, whereby pooled enriched cells are prepared, wherein the pooled enriched cells have a viability rate greater than that of the human primary cells from at least one of the two or more donors;
wherein a total concentration of a density gradient solution in the second density gradient separation is lower than a total concentration of a density gradient solution in the first density gradient separation.

According to another embodiment, a method of preparing pooled enriched cells is provided. The method comprises:
(a) obtaining primary cells from two or more donors;
(b) removing dying or dead cells from the primary cells from each of the two or more donors by a first density gradient separation whereby density gradient treated cells are generated;
(c) cryopreserving the density gradient treated primary cells to obtain cryopreserved density gradient treated primary cells from each of the two or more donors;
(d) thawing the cryopreserved density gradient treated primary cells from each of the two or more donors whereby thawed density gradient treated primary cells from each of the two or more donors are generated;
(e) combining the primary cells from the two or more donors, whereby combined primary cells are generated;
(f) removing dying or dead cells from the combined human primary cells by a second density gradient separation, whereby combined separated cells are generated;
(g) cryopreserving the combined separated cells, whereby cryopreserved combined separated cells are generated;
(h) thawing the cryopreserved combined separated cells, whereby thawed combined separated cells are generated; and
(i) removing dying or dead cells from the thawed combined separated cells by a third density gradient separation, whereby pooled enriched cells are prepared, wherein the pooled enriched cells have a viability rate greater than that of the human primary cells from at least one of the two or more donors;
wherein a total concentration of a density gradient solution in the third density gradient separation is equal to or higher than a total concentration of a density gradient solution in the second density gradient separation.

According to yet another embodiment, another method for preparing enriched cells is provided. This embodiment comprises:
(a) obtaining primary cells from a donor;
(b) removing dying or dead cells from the primary cells by a first density gradient separation, whereby separated cells are generated;
(c) cryopreserving the separated cells, whereby cryopreserved separated cells are generated;
(d) thawing the cryopreserved separated cells, whereby thawed separated cells are generated; and
(e) removing dying or dead cells from the thawed separated cells by a second density gradient separation, whereby enriched cells are prepared, wherein the enriched cells have a is viability rate greater than that of the primary cells from the donor,
wherein a total concentration of a density gradient solution in the second density gradient separation is lower than a total concentration of a density gradient solution in the first density gradient separation.

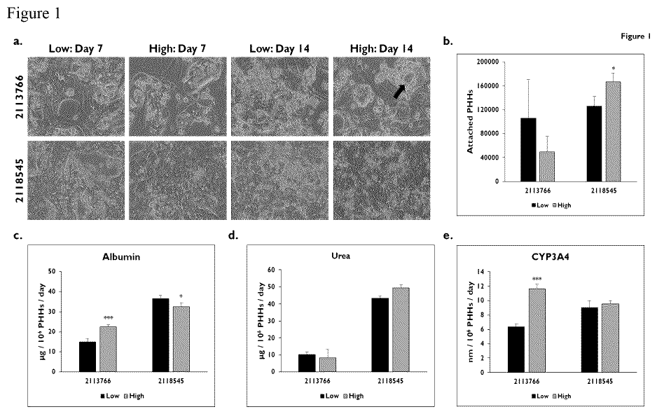

FIG. 1 shows PHHs from type 2 diabetes mellitus (T2DM) donors enriched in either low or high Percoll® exhibit differences in morphology and function. (a) Morphology of T2DM lots 2113766 (top row) and 2118545 (bottom row) on days 7 and 14 after enrichment in low and high Percoll®. Arrow indicates vacuole. Magnification=20×. Scale bar=50 μm. (b) Attached number of PHHs on day 14 after enrichment in low (black bars) and high (grey bars) Percoll®. n=5 images per condition for each donor lot. (c) Albumin and (d) urea levels normalized to number of attached PHHs on day 14 after enrichment in low (black bars) and high (grey bars) Percoll®. n≥2 samples per condition. (e) CYP3A4 activity on day 14 from PHHs enriched in low (black bars) and high (grey bars) Percoll®. n=6 samples per condition. Error bars represent standard deviation. *p<0.05 and ***p<0.001 to low Percoll®.

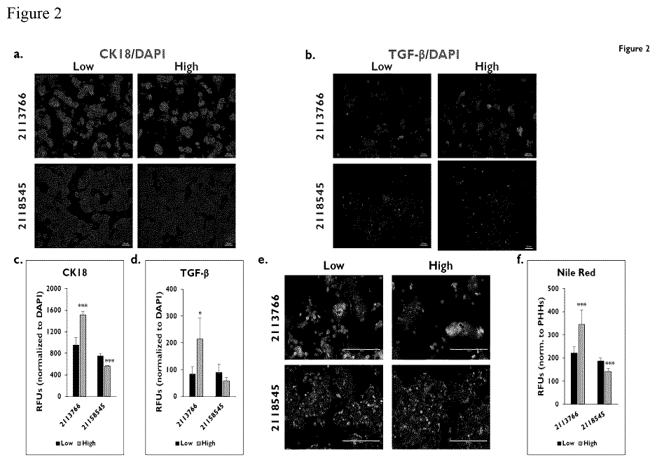

FIG. 2 shows Percoll® enriched PHHs have stronger expression of fibrotic markers compared to high Percoll® enriched PHHs from type 2 diabetes mellitus (T2DM) donors. Marker expression of fibrotic proteins (a) CK18 (red) and (b) TGF-β (red) plus DAPI staining (blue) on day 14 in T2DM lots 2113766 (top row) and 2118545 (bottom row) after enrichment in low (left column) and high (right column) Percoll®. Magnification=10×. Scale bar=100 μm. Quantification of (c) CK18 and (d) TGF-β marker expression in PHHs after enrichment in low (black bars) and high (grey bars) Percoll®. n=5 images per condition for each donor. (e) Nile red staining of T2DM lots 2113766 (top row) and 2118545 (bottom row) on day 14 after enrichment in low (left column) and high Percoll® (right column). Magnification=20×. Scale bar=200 μm. (f) Quantification of fluorescence from Nile Red staining in relative fluorescence units (RFUs) after PHHs have been enriched in low (black bars) and high (grey bars) Percoll®. n≥5 images per condition for each donor lot. Values have been normalized to number of attached is PHHs for each donor lot and condition. Error bars represent standard deviation. *p<0.05 and ***p<0.001 to low Percoll®.

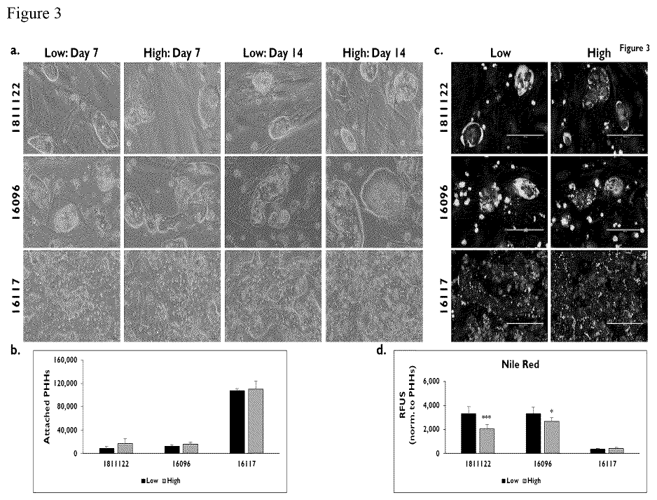

FIG. 3 shows diseased PHHs enriched in low Percoll® have increased lipid accumulation compared to diseased PHHs enriched in high Percoll®. (a) Morphology of diseased donor lots 1811122 (top row) and 16096 (middle row) and normal donor lot 16117 (bottom row) on days 7 and 14 after enrichment in low and high Percoll®. Magnification=20×. Scale bar=50 μm. (b) Number of attached PHHs on day 14 after enrichment in low (black bars) and high (grey bars) Percoll®. n=5 images per condition for each donor lot. (c) Staining of lipids by Nile Red in diseased lots 1811122 (top row) and 16096 (middle row) and normal lot 16117 (bottom row) on day 14. PHHs were enriched in low (left column) and high Percoll® (right column). Magnification=20×. Scale bar=200 μm. (d) Quantification of fluorescence from Nile Red staining in relative fluorescence units (RFUs) after enrichment in low (black bars) and high (grey bars) Percoll®. n≥5 images per condition for each donor lot. Values are normalized to number of attached PHHs for each lot and condition. Error bars represent standard deviation. *p<0.05 and ***p<0.001 to low Percoll®.

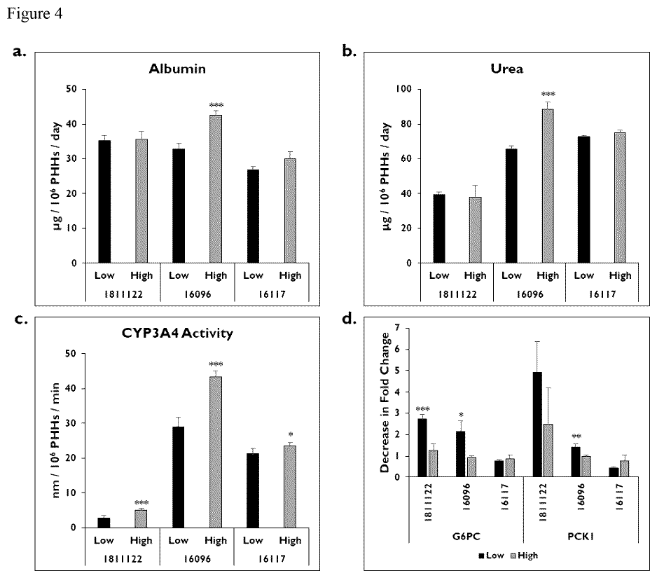

FIG. 4 shows CYP3A4 activity decreases in diseased lots enriched in low Percoll®. (a) Albumin and (b) urea levels on day 14 normalized to attached PHHs in diseased lots 1811122 and 16096, and normal lot 16117 enriched in low (black bars) and high (grey bars) Percoll®. ***p<0.001 to low Percoll®. (c) Baseline CYP3A4 activity on day 14 normalized to attached PHHs after enrichment in low (black bars) and high (grey bars) Percoll®. *p<0.05 and ***p<0.001 to low Percoll®. (d) G6PC and PCK1 gene expression represented as decrease in fold change on day 14 in lots enriched in low (black bars) and high (grey bars) Percoll®. Data normalized to PHHs enriched in high Percoll®. Error bars represent standard deviation. *p<0.05, **p<0.01, and ***p<0.001 to high Percoll®.

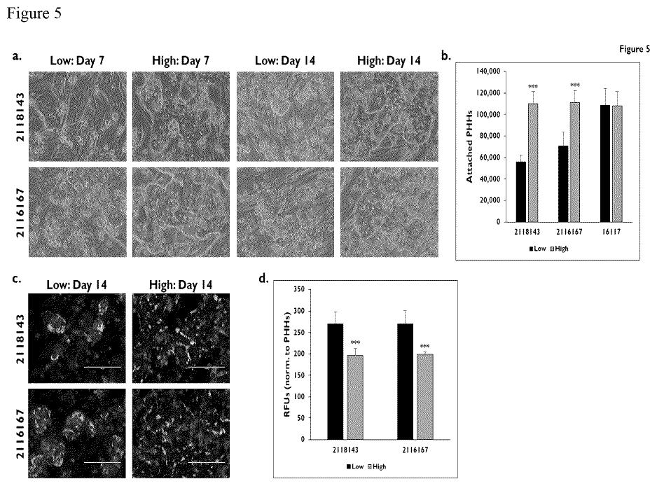

FIG. 5 shows diseased PHHs have lower attachment after enrichment in low Percoll® compared to diseased PHHs enriched in high Percoll®. (a) Morphology of diseased lots 2118143 (top row) and 2116167 (bottom row) on days 7 and 14 after enrichment in low and high Percoll®. Magnification=20×. Scale bar=50 μm. (b) Number of attached PHHs on day 14 after enrichment in low (black bars) and high (grey bars) Percoll®. n=5 images per condition is for each donor lot. (c) Nile Red staining on hepatocyte lots 2118143 (top row) and 2116167 (bottom row) on day 14 in PHHs enriched in low and high Percoll®. Magnification=20×. Scale bar=200 μm. (d) Quantification of fluorescence in relative fluorescence units (RFUs) from Nile Red staining when PHHs were enriched in low (black bars) and high (grey bars) Percoll®. n≥3 images per condition for each donor lot. Values have been normalized to number of attached PHHs for each lot and condition. Error bars represent standard deviation. ***p<0.001 to low Percoll®.

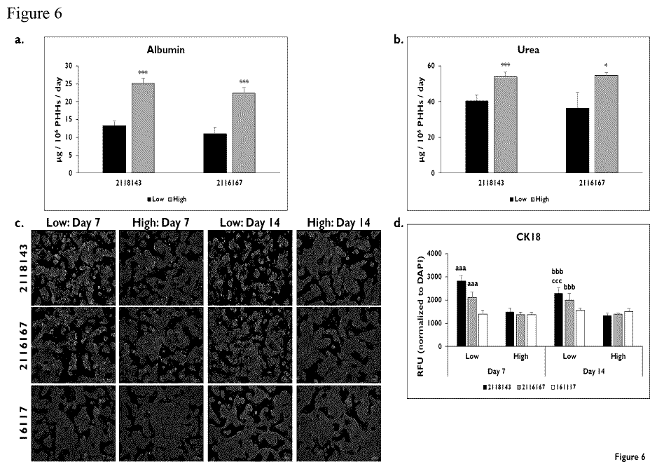

FIG. 6 shows cytokeratin 18 expression is higher in diseased lots enriched in low Percoll®. (a) Albumin and (b) urea levels on day 14 from diseased lots 2118143 and 2116167 after enrichment in low (black bars) and high (grey bars) Percoll®. n=2 samples per condition. (c) CK18 protein expression (red) plus DAPI (blue) on days 7 and 14 in lots 2118143 (top row), 2116167 (middle row), and 16117 (bottom row) after enrichment in low and high Percoll®. Magnification=10×. Scale bar=100 μm. (d) CK18 quantification in relative fluorescence units (RFU) normalized to DAPI number on days 7 and 14 in lots 2118143 (black bars), 2116167 (grey bars), and 16117 (white bars) after enrichment in low and high Percoll®. n=5 images per condition for each donor lot. Error bars represent standard deviation. ^{aaa}p<0.001 to high Percoll® on day 7. ^{bbb}p<0.001 to high Percoll® on day 14. ^{ccc}p<0.001 to low Percoll® on day 7.

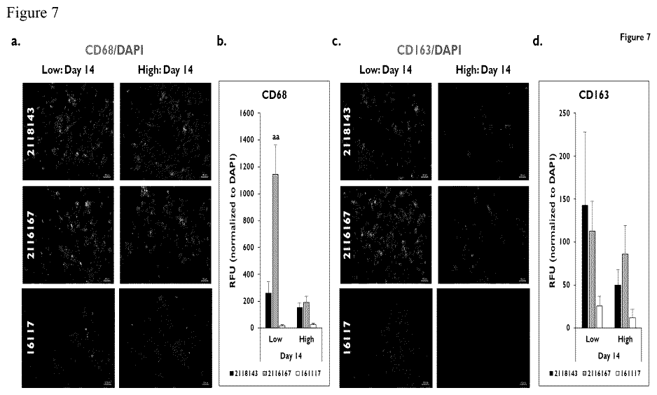

FIG. 7 shows fibrotic PHHs enriched in low Percoll® have increased CD68 but not CD163 marker expression compared to those enriched in high Percoll® on day 14. (a) CD68 (green) marker expression plus DAPI (blue) on day 14 in lots 2118143 (top row), 2116167 (middle row) and 16117 (bottom row) after being enriched in low and high Percoll®. (b) CD68 quantification in relative fluorescence units (RFU) normalized to DAPI number on day 14 in lots 2118143 (black bars), 2116167 (grey bars), and 16117 (white bars) after enrichment in low and high Percoll®. (c) CD163 (green) marker expression plus DAPI (blue) on day 14 in lots 2118143 (top row), 2116167 (middle row) and 16117 (bottom row) after being enriched in low and high Percoll®. (d) Quantification of CD163 fluorescence in relative fluorescence units (RFU) normalized to DAPI number on day 14 in lots 2118143 (black bars), 2116167 (grey bars), and 16117 (white bars) after enrichment in low and high Percoll®. Error bars represent standard deviation. ^{aa}p<0.01 on day 14 to high Percoll®.

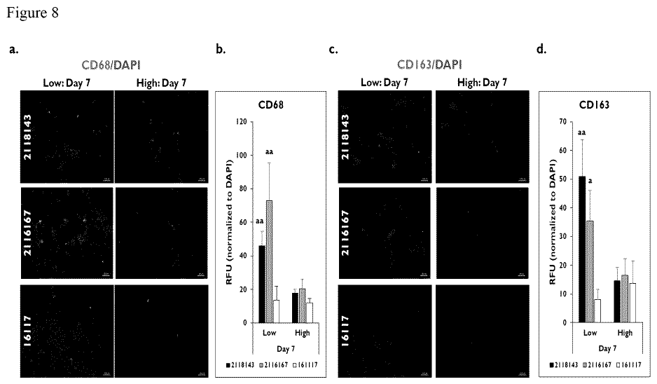

FIG. 8 shows CD68 and CD163 marker expression is higher in fibrotic PHHs enriched in low Percoll® versus fibrotic PHHs enriched in high Percoll® on day 7. (a) CD68 (green) marker expression plus DAPI (blue) on day 7 in lots 2118143 (top row), 2116167 (middle row) and 16117 (bottom row) after being enriched in low and high Percoll®. Magnification=10×. Scale bar=100 μm. (b) CD68 quantification in relative fluorescence units (RFU) normalized to DAPI number on day 7 in lots 2118143 (black bars), 2116167 (grey bars), and 16117 (white bars) after enrichment in low and high Percoll®. n=5 images per condition for each donor lot. (c) CD163 (green) marker expression plus DAPI (blue) on day 7 in lots 2118143 (top row), 2116167 (middle row) and 16117 (bottom row) after being enriched in low and high Percoll®. Magnification=10×. Scale bar=100 μm. (d) Quantification of CD163 fluorescence in relative fluorescence units (RFU) normalized to DAPI number on day 7 in lots 2118143 (black bars), 2116167 (grey bars), and 16117 (white bars) after enrichment in low and high Percoll®. n=5 images per condition for each donor lot. Error bars represent standard deviation. ^{a}p<0.05 and ^{aa}p<0.01 on day 7 to high Percoll®.

### DETAILED DESCRIPTION OF THE INVENTION

The present invention provides pooled cells prepared from mammalian cells, such as human primary cells, for example, liver cells such as hepatocytes, lung cells such as AT2 lung cells, thyrocytes and pancreatic islets, and preparation and uses thereof. The present invention also provides cells from a single donor and preparation and use thereof. The inventors have found that certain preparation methods are capable of improving the viability and heterogeneity of pooled cells (i.e., cells pooled from two or more donors). These preparation methods also improve the viability of batches of cells from single donors, in particular, cells having certain desirable phenotypes, such as certain liver diseases, certain thyroid diseases, or certain lung diseases. According to some embodiments, the type of cells that may be usefully subjected to this method may include epithelial, endothelial, stromal, mesenchymal, ectodermal, immune, and combinations thereof.

According to some embodiments, there are two applications of the present methods that are distinct but may be overlapping. The first application is pooling multiple donor cells to generate pooled cell inventory. In this method, a density gradient separation is applied during isolation and then a density gradient separation is then applied during the generation of the pooled cells for final use. A second application of the method is adjusting the stringency of the density gradient separation to modulate the phenotypic heterogeneity of the final population, i.e., fine-tuning heterogeneity and the potential for observation of disease-like or cell type specific outcomes through adjustment of the density of the density gradient separation.

The invention is based on the inventors' surprising discovery of significant improvement of the viability and heterogeneity rate of human primary cells, such as liver cells or thyrocytes or AT2 lung cells or pancreatic islets, prepared by pooling the previously-cryopreserved cells from multiple donors and removing dying or dead cells by three density gradient separations. Pooled hepatocytes may go through three density gradient separations as follows. One during the isolation process, one during the pooling process, and one during the post-thaw process. The inventors have also found that this method of utilizing three density gradient steps also improves the viability rate of cells from a single donor, in particular, cells that have been obtained from donors having certain desirable phenotypes, especially diseases of the liver, thyroid or lungs. The inventors also have found that by utilizing the three density gradient separations, the cells may be enriched in certain desirable disease types. In other words, products comprising pooled cells or single donor cells are useful for many applications, including testing a pharmaceutical substance, drug metabolism, drug transport or drug toxicity, and preparing cell models such as a hepatitis B virus (HBV) infected hepatocyte culture model. Other diseased cell models may include but are not limited to non-alcoholic fatty liver disease (NAFLD), non-alcoholic steatohepatitis (NASH), type 2 diabetes mellitus (T2DM), benign simple steatosis, fibrosis, cirrhosis, and hepatocellular carcinoma (HCC). If thyroid cells are prepared, the cell model may include hyperthyroidism, hypothyroidism, Hashimoto's thyroiditis, Graves' disease, goiter, thyroid nodules, thyroid tumors, or thyroid cancer. If AT2 lung cells are prepared, the cell model may include lung diseases such as idiopathic pulmonary fibrosis (IPF) which may include a pro-fibrotic phenotype, lung adenocarcinoma (LUAD), acute lung injury (ALI) (e.g. pneumonia, inhalation of toxic substances, fat embolism, viral infection of the lungs), acute respiratory distress syndrome (ARDS), hyperplasia, or chronic obstructive pulmonary disease (COPD). If pancreatic islets are prepared, the cell model may include pancreatitis, pancreatic cancer, or type 1 or type 2 diabetes is mellitus (T1DM or T2DM).

The present invention also provides methods of preparing the cells (pooled or single donor), including first and second pre-freezing density gradient treatments and a third post-thaw density gradient treatment. The first, second and third density gradient treatments may have different density gradients from each other. For example, the concentration of the density gradient may be higher in the first density gradient treatment than in the second density gradient treatment, and the second density gradient treatment may have a concentration lower than that of the third density gradient treatment. The three different density gradient treatments may be especially useful when preparing pooled cells.

For example, the preparation of the pooled cells may proceed as follows.

The cells (e.g. liver cells such as hepatocytes, thyrocytes, AT2 lung cells, or pancreatic islets) may be isolated and subjected to density gradient and processing (e.g., in Percoll®) (P1) to prepare cryopreserved cells, such as hepatocytes. Then, thaw the cell vials from multiple donors, optionally pool the cells, then use an equal or lower concentration density gradient (such as Percoll®) (P2) for removal of cell debris, and then cryopreserve the cells again. Finally, at the point of using the cells for an assay(s), thaw the hepatocytes and use an equal or higher concentration density gradient treatment (such as Percoll®) (P3) for removal of cell debris. According to an embodiment of this method, P3 is equal to or greater than P2, and cell viability>70%. This method may provide enhanced heterogeneity of the pooled cells, if the optional pooling step is performed. Thus, the cell viability at the end of P2 (prior to second cryogenic freezing step) is <70%.

According to another embodiment of the method, the method is focused on preparing cells that preserve certain desirable phenotypes, i.e. diseased cells (e.g. diseased liver cells, diseased thyrocytes, diseased AT2 lung cells, or diseased pancreatic islets). The steps of this method are as follows. First, isolate the cells and subject them to density gradient treatment (e.g. Percoll®) (P1) and processing to prepare cryopreserved cells. At the point of using the cells for an assay(s), the cells are thawed and a lower concentration density gradient treatment (e.g. Percoll®) (P2) is used for removal of cell debris. According to an embodiment of this method, P2 is lower than P1 and the method provides a higher percentage of diseased cell populations and representation of disease phenotype compared to a method that does not subject the cells to a is post-thaw density gradient treatment at all, or if the post-thaw density gradient step is not of lower concentration than the density gradient treatment prior to cryopreservation. This method may be used for the pooled or single donor cell populations.

According to a particular embodiment, if Percoll® is used for the density gradient separations, about 27% Percoll® may be used for the first density gradient separation and then about 27% or about 35% Percoll® may be used for the second density gradient.

The term “primary cell” as used herein refers to a cell from a donor that has not been cultured or has been cultured for not more than a predetermined number of passages (e.g., 0, 1, 2, 3, 4 or 5 passages).

The term “liver cell” as used herein, means any cell from the liver, including parenchymal cells, such as hepatocytes, and non-parenchymal liver cells such as endothelial cells, fibroblasts, hepatic stellate cells, and immune cells. Other liver-derived epithelial cells, such as bile duct epithelial cells, may be isolated and pooled.

The term “hepatocyte” as used herein refers to a cell originally from a liver. The hepatocyte may be a primary hepatocyte or a cell derived from a primary hepatocyte. An hepatocyte may have a biomarker of a plasma protein such as albumin, transferrin, transthyretin or α-1-antitrypsin, or a surface or intracellular protein such as CK8, CK18, cluster of differentiation 81 (CD81), or asialoglycoprotein receptor (ASPGR).

The terms “thyrocyte,” “thyroid follicular cell,” and “thyroid epithelial cell” are used herein interchangeably and refer to an epithelial cell originally from a thyroid gland. The thyrocyte may be a primary thyrocyte or a cell derived from a primary thyrocyte. A thyrocyte may have an epithelial marker, for example, cytokeratin 7 (CK7), thyroglobulin (TG), epithelial cellular adhesion molecule (EpCAM), thyroid stimulating hormone (TSH) receptor, or thyroperoxidase (TPO) receptor.

The term “AT2 lung cell” as used here refers to a cell originally from the lungs and include biomarkers such as HTII-280, surfactant protein C (SPC), or lysosome-associated membrane glycoprotein 3 (LAMP3).

The term “pancreatic islet” as used here refers to a cell originally from the pancreas and includes biomarkers such as insulin, glucagon, somatostatin, pancreatic polypeptide, and ghrelin. The term, “epithelial cells”, as used here means cells that arise from the cellular, avascular layer covering all of the free surfaces, cutaneous, mucous, and serous, including the glands and structures derived therefrom. Epithelial cells may be simple squamous, simple cuboidal, simple columnar, ciliated columnar, pseudostratified ciliated columnar with goblet cells, stratified squamous, or transitional. Non-limiting examples of epithelial cells include thyroid cells, pancreas islet cells, kidney cells, stomach cells, bladder and urinary tract cells, portions of the respiratory tract.

The term “pooled cells” as used herein refers to a mixture of cells originally from two or more donors.

The term “single donor cells” as used herein refers to a mixture of cells originally from a single donor.

The term “pooled hepatocytes” as used herein refers to a mixture of hepatocytes originally from two or more donors.

The term “pooled thyrocytes” as used herein refers to a mixture of thyrocytes originally from two or more donors.

The term “pooled AT2 lung cells” as used herein refers to a mixture of AT2 lung cells originally from two or more donors.

The term “pooled pancreatic islets” as used herein refers to a mixture of pancreatic islets originally from two or more donors.

The term “single donor hepatocytes” as used herein refers to a mixture of hepatocytes originally from a single donor.

The term “single donor thyrocytes” as used herein refers to a mixture of thyrocytes originally from a single donor.

The term “single donor AT2 lung cells” as used herein refers to a mixture of AT2 lung cells originally from a single donor.

The term “single donor pancreatic islets” as used herein refers to a mixture of pancreatic islets originally from a single donor.

The term, “enriched cells”, whether pooled or from a single donor, means cells that have undergone at least one of the preparation methods as described herein. Such cells may be is enriched in such attributes as viability, populations of diseased phenotypes, or heterogeneity (if pooled).

The term “donor” used herein refers to a living mammal. The mammal may be a human, a cow, a pig, a dog, a cat, a non-human primate, a rodent such as a rat or mouse, a horse, a goat, a sheep, or a deer. The donor may be healthy or suffer from a disease or disorder.

“Density gradient separation” as used herein means the density range of the gradient medium encompasses all densities of the sample particles. Each particle will sediment to an equilibrium position in the gradient where the gradient density is equal to the density of the particle (isopycnic position). Thus, in this type of separation, the particles are separated solely based on differences in density, irrespective of size. The density gradients utilized herein may be continuous or discontinuous. The density gradient may be linear, convex, concave, isokinetic, or linear-logarithmic. The gradients may be formed by centrifugation, overlayering, or underlayering of the gradient media for example. Other methods of preparing a density gradient include diffusion of discontinuous gradients, gradient mixing chamber, or a gradient mixer. Non-limiting examples of suitable density gradient media include but are not limited to heavy metal salts, carbohydrates, iodinated compounds and colloidal silica gels. Specific such materials are Histopaque, sodium metrizoate, CsCl, Cs₂O₄, D₂O, Ficoll®, Iodixanol (Visipaque™ or OptiPrep™), Nycodenz®, sorbitol, sucrose, and Percoll®. The media and concentration thereof may be selected based on the range of densities needed for the desired separation. It also is understood that the density gradient is affected by the centrifuge speed combined with the concentration of the density gradient media. Generally, higher centrifuge speeds lead to greater slope in the density gradient, meaning that there is a bigger difference between the highest and lowest density in the gradient.

The term “separated cells” as used herein means cells that have undergone at least one density gradient separation step and reference to the cells that were retained after the completion of the density gradient treatment.

As used herein, the terms “high density gradient medium” and “low density gradient medium” or “high Percoll®” and “low Percoll®” are relative to each other in reference to two or more density gradient separation steps in a method. As is known in the art, the target % (v/v) of the density gradient medium in any density separation method is affected by the type and condition of cells to be separated, the speed of the centrifuge and the type of density gradient medium that is used. While Percoll® is used as an example of a density gradient medium herein that may be used in any embodiment of the disclosed method, any density gradient medium discussed herein may be used in the inventive method. Non-limiting examples are as follows.

Ficoll® is a synthetic polymer made from the co-polymerization of sucrose and epichlorohydrin. It is a highly branched, uncharged polymer with a molecular weight of around 400,000.

Iodixanol (5-[acetyl-[3-[acetyl-[3,5-bis(2,3-dihydroxypropylcarbamoyl)-2,4,6-triiodo-phenyl]amino]-2-hydroxy-propyl]amino]-N,N′-bis(2,3-dihydroxypropyl)-2,4,6-triiodo-benzene-1,3-dicarboxamide) may be used to form density gradients.

Nycodenz is a non-ionic tri-iodinated derivative of benzoic acid with three aliphatic hydrophilic side chains. The systematic name of Nycodenz is 5-(N-2, 3-dihydroxypropylacetamido)-2, 4, 6-tri-iodo-N, N′-bis (2, 3 dihydroxypropyl) isophthalamide. Nycodenz has a molecular weight of 821 and a density of 2.1 g/ml.

Percoll® is a tool for efficient density separation in cell biology. It is used for the isolation of cells, organelles, and/or viruses by density centrifugation. Percoll® includes colloidal silica particles of 15-30 nm diameter (23% w/w in water) which have been coated with polyvinylpyrrolidone (PVP). Percoll® has a density of 1.13 g/ml.

Histopaque is a density gradient cell separation medium of Ficoll® and sodium diatrizoate.

Sodium metrizoate is sodium N-[3-carboxy-2,4,6-triiodo-5-(N-methylacetamido)phenyl]ethanecarboximidate.

Chronic liver disease is a significant global public health concern, representing a growing cause of mortality and morbidity worldwide. While the onset of liver disease is multi-faceted, non-alcoholic fatty liver disease (NAFLD) remains the most common driver. An estimated 38% of the adult population globally is currently afflicted with NAFLD, resulting in a substantial is healthcare and economic burden. NAFLD encompasses an inflammatory spectrum ranging from benign simple steatosis to non-alcoholic steatohepatitis (NASH), eventually progressing to fibrosis, liver cirrhosis, and hepatocellular carcinoma (HCC). As the prevalence of NAFLD is projected to steadily increase over the next decades, potentially rising to 55% of the global population by 2040, it is imperative to develop comprehensive liver injury models to better understand disease pathogenesis and to support the design of effective pharmacological therapies.

Animal disease models, primarily murine-based, are widely utilized to study the metabolic pathways and factors underlying NAFLD development and progression. While these models can incorporate genetic and dietary triggers for disease onset, they often do not accurately reproduce human disease due to the use of artificial induction regimes. They also fail to fully capture the distinct features of NAFLD, and interspecies differences from preclinical findings are often unsuccessful at reliably predicting human-relevant pathogenesis. The limitations of existing animal models further highlight the need for improved human liver model systems that maintain hepatic cells from diseased tissues and better retain key elements of human NAFLD.

The TruVivo® cell system, an all-human cell-based triculture system, was developed to provide a robust 2D in vitro system that better mimics the in vivo hepatic microenvironment. Comprised of three different human cell types, including cryopreserved primary human feeder cells (FCs), comprised of stromal cells and endothelial cells, and cryopreserved primary human hepatocytes (PHHs), this system maintains stable albumin and urea production, and cytochrome P450 3A4 (CYP3A4) activity for at least two weeks. This platform has been further extended to incorporate PHHs selected from tissue donors with known medical history of NAFLD to generate a diseased TruVivo® cell system. PHHs derived from diseased liver tissues sustain a characteristic fatty liver disease phenotype for extended culture periods, including decreased hepatocyte functionality, significant basal lipid accumulation, altered lipogenic responses and increased pro-inflammatory cytokine production. In addition, the diseased TruVivo® cell system can be modulated to support the investigation of early-stage steatosis development, as well as is later stage disease progression. The presence and longevity of these disease-relevant attributes suggest the diseased TruVivo® cell system is a promising tool for advancement of models across the spectrum of liver disease.

To best predict toxicology and safety as a preclinical hepatic system, an improved understanding of how the diseased PHH phenotype manifests under varying culture conditions is needed. In these studies, PHHs in the TruVivo® cell system were cultured following post-thaw processing in low and high percentage Percoll®-based thawing medium. Percoll® colloidal silica medium is used for density-based cell separation, including endothelial, epithelial and fibroblastic cells, allowing for separation and/or enrichment of unique cell populations. In low-density Percoll® conditions, diseased PHHs had lower attachment and increased fat accumulation on day 14 of culture as compared to higher percentage Percoll® thawing medium, while hepatic functions of albumin and urea production and CYP3A4 activity were also impacted. Expression of the fibrotic markers cytokeratin 18 (CK18) and transforming growth factor beta (TGF-0) and the macrophage markers CD68 and CD163 were also highest when diseased PHHs were enriched in low percentage versus high percentage Percoll®, as was expression of genes related to production of enzymes that are critical to gluconeogenesis. In a subset of PHHs derived from T2DM donors, expression of fibrotic markers was altered following low or high Percoll® enrichment. Taken together, treatment with either a low or high Percoll® thawing medium can support a sustained diseased phenotype with differences in attachment, functionality, lipogenesis, and marker expression in the TruVivo® cell system.

Thyroid disease is also of concern worldwide and the present method may be used to provide model thyroid cell populations (especially thyrocytes) that have high viability, may have a high proportion of certain desirable phenotypes, and may also have a high degree of heterogeneity in terms of age, race, and disease, for example. The same is true for AT2 lung cells and pancreatic islets.

The donor or donors may be an animal, such as a human, of any age. The donor may be a juvenile, who is between about 28 days old and about 18 years old. The donor may be a neonate, who is under about 28 days old. The donor may be under about 1, 2, 3, 4, 5, 6, 7, 14, 21 or 28 days old, or about 0-7, 0-14, 0-21, 0-28, 7-14, 7-21, 7-28, 14-21, 14-28 or 21-28 days old. The donor may be older than about 28 days and younger than about 1, 2, 3, 4, 5, 6, 7, 8, 9, 10, 11, 12, is 13, 14, 15, 16, 17 or 18 years old, or about 1-18, 2-18, 3-18, 4-18, 5-18, 6-18, 7-18, 8-18, 9-18, 10-18, 11-18, 12-18, 13-18, 14-18, 15-18, 16-18 or 17-18 years old.

The term “viable cell” as used herein refers to a cell that is metabolically active, living, developing, and capable of reproducing.

The term “viability rate” as used herein refers to a percentage of viable cells in a mixture of cells.

The term “dying cell” as used herein refers to a cell committed to cell death, for example, apoptosis. The dying cell may have compromised cell membrane integrity such that the cell's buoyant density is impacted. A dying cell may still be viable.

The term “dead cell” as used herein refers to a cell that is not living, developing and/or reproducing.

The term “cryo-damaged cell” as used herein refers to a cell having damage caused by an extreme cold treatment. The cryo-damaged cells may have a declined viability rate and/or biological function. Extreme cold treatment may be a treatment with liquid nitrogen or argon gas. The cryo-damaged cells may have a biomarker for a non-viable cell, for example, trypan blue positive or propidium iodide positive, or a biomarker for a caspase activity. The cryo-damaged cells appear different under the microscope compared to non-cryo-damaged cells. A non-viable cell may still be alive but will not survive plating and attach and proliferate. Such a cell would therefore be considered non-viable.

The term “functional stability” or “functionally stable” used herein refers to the maintenance of a biological function by cells over a predetermined period of time. The functional stability may be characterized by a decline of a biological function of the cells after a predetermined period of time. Cells may be deemed functionally stable for a biological function if the cells maintain the biological function with a decline less than about 5%, 10%, 15%, 20%, 25%, 30%, 35%, 40%, 45%, 50%, 60%, 70%, 80%, 90%, 95% or 99%, optionally with a deviation no more than about 5, 10, 15, 20 or 25%, after a predetermined period of time (e.g., about 3, 4, 5 or 6 days or 1, 2, 3, 4, 5, 6, 7, 8, 9 or 10 weeks, or about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour).

The term “culture longevity” used herein refers to the lifetime of cultured cells that remain viable and/or functionally stable. At least about 70%, 75%, 80%, 85%, 90%, 95%, 99% or 100% of the cells may remain viable and/or functionally stable for a predetermined period of time (e.g., about 3, 4, 5 or 6 days or 1, 2, 3, 4, 5, 6, 7, 8, 9 or 10 weeks, or about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour). The viable and/or functionally stable cultured cells may form a monolayer, an aggregate, or a combination thereof. The viable and/or functionally stable cultured cells may remain in suspension. The monolayer may be in contact with a surface. The surface may be coated with, for example, an extracellular matrix or a hydrogel component.

The term “aggregate” as used herein refers to a cluster of two or more cells. Each cell is in direct contact with at least another cell in the cluster.

The term “density gradient separation” as used herein refers to separation of different cells in a density gradient medium based on their different densities.

The term “stringent” as used herein refers to a density gradient that has a higher density/specific gravity.

A method for preparing enriched pooled cells is provided. The preparation method comprises obtaining primary cells from two or more donors, and combining the primary cells from the two or more donors to generate combined primary cells. The preparation method also comprises removing dying or dead cells from the combined primary cells by a first density gradient separation to generate combined separated cells. The preparation method further comprises cryopreserving the combined separated cells to generate cryopreserved combined separated cells; thawing the cryopreserved combined separated cells to generate thawed combined separated cells; and removing dying or dead cells from the thawed combined separated cells by a second density gradient separation, whereby enriched pooled cells are prepared. The enriched pooled cells have a viability rate greater than that of the primary cells from at least one of the two or more donors.

According to another embodiment of the preparation method, the cells are not pooled but are instead obtained from a single donor. In this embodiment, the method comprises obtaining cells from a single donor and then comprises removing dying or dead cells from the primary is cells by a first density gradient separation to generate separated cells. The preparation method further comprises cryopreserving the separated cells to generate cryopreserved separated cells; thawing the cryopreserved separated cells to generate thawed separated cells; and removing dying or dead cells from the thawed separated cells by a second density gradient separation, whereby enriched cells are prepared. The enriched cells have a viability rate greater than that of the primary cells from the donor.

According to an embodiment of the preparation method, the pooled enriched cells or the enriched cells from a single donor may have a viability rate greater than that of the primary cells from the two or more donors. The pooled enriched cells or the enriched cells from a single donor may have a viability rate at least about 5%, 10%, 20%, 30%, 40% or 50% greater than that of the primary human cells from the two or more donors or from a single donor, respectively. The pooled cells may have a viability rate at least about 5%, 10%, 20%, 30%, 40% or 50% greater than that of the primary human cells from at least one of the two or more donors, or from a single donor, respectively.

Another embodiment for preparing pooled enriched cells is also provided. The embodiment of the preparation method comprises obtaining primary cells from two or more donors and removing dying or dead cells from the primary cells from each of the two or more donors by a first density gradient separation such that separated cells from each of the two or more donors are generated. The embodiment of the preparation method also comprises combining the separated cells from the two or more donors to generate combined separated cells. The embodiment of the preparation method further comprises cryopreserving the combined separated cells to generate cryopreserved combined separated cells; thawing the cryopreserved combined separated cells to generate thawed combined separated cells; and removing dying or dead cells from the thawed combined separated cells by a second density gradient separation, whereby enriched pooled cells are prepared. The enriched pooled cells have a viability rate greater than that of the human primary cells from at least one of the two or more donors.

According to an embodiment of the preparation method, the pooled enriched cells may have a viability rate greater than that of the primary cells from the two or more donors. The pooled enriched cells may have a viability rate at least about 5%, 10%, 20%, 30%, 40% or 50% greater than that of the primary cells from the two or more donors. The pooled enriched cells may have a viability rate at least about 5%, 10%, 20%, 30%, 40% or 50% greater than that of the primary human cells from at least one of the two or more donors.

Another embodiment of the method for preparing pooled enriched cells is provided. The method comprises obtaining primary cells from two or more donors and removing dying or dead cells from the primary cells from each of the two or more donors by a first density gradient separation whereby density gradient treated cells are generated. This embodiment of the method also comprises cryopreserving the density gradient treated primary cells to obtain cryopreserved density gradient treated primary cells from each of the two or more donors. The embodiment of the method also comprises thawing the cryopreserved density gradient treated primary cells from each of the two or more donors whereby thawed density gradient treated primary cells from each of the two or more donors are generated and then combining the primary cells from the two or more donors, whereby combined primary cells are generated. This embodiment of the method then comprises removing dying or dead cells from the combined primary cells by a second density gradient separation, whereby combined separated cells are generated and then cryopreserving the combined separated cells, whereby cryopreserved combined separated cells are generated. This embodiment of the method also comprises thawing the cryopreserved combined separated cells, whereby thawed combined separated cells are generated and removing dying or dead cells from the thawed combined separated cells by a third density gradient separation, whereby enriched pooled cells are prepared, wherein the enriched pooled cells have a viability rate greater than that of the primary cells from at least one of the two or more donors. In this embodiment of the method, a concentration of a density gradient solution in the third density gradient separation is equal to or higher than a density gradient solution in the second density gradient separation.

According to any of the methods or products disclosed herein, the cells may be animal cells, particularly mammal cells, such as human cells.

According to the disclosed methods, the primary cells from a single donor or the two or more donors may be hepatocytes, and the pooled cells may be pooled hepatocytes. The single donor or at least one of the two or more donors may be a human having a healthy liver or a liver with a disease or a condition, for example, microsteatosis, non-alcoholic steatohepatitis (NASH), non-alcoholic fatty liver disease (NAFLD) or hepatitis (e.g., A, B, C, D and E). Other diseased is cell models may include but are not limited to type 2 diabetes mellitus (T2DM), fibrosis, cirrhosis, and hepatocellular carcinoma (HCC).

According to the disclosed methods, the primary cells from the two or more donors may be thyrocytes, and the pooled cells may be pooled thyrocytes. At least one of the two or more donors may be a human having a healthy thyroid or a thyroid with a disease or disorder, for example, hyperthyroidism, hypothyroidism, Hashimoto's thyroiditis, Graves' disease, goiter, thyroid nodules, thyroid tumors, or thyroid cancer.

According to the disclosed methods, the human cells from the two or more donors may be AT2 lung cells, and the pooled cells may be pooled AT2 lung cells. At least one of the two or more donors may be a human having a healthy lung or a lung with a disease or disorder, for example, idiopathic pulmonary fibrosis (IPF) which may include a pro-fibrotic phenotype, lung adenocarcinoma (LUAD), acute lung injury (ALI) (e.g. pneumonia, inhalation of toxic substances, fat embolism, viral infection of the lungs), acute respiratory distress syndrome (ARDS), hyperplasia, or chronic obstructive pulmonary disease (COPD).

According to the disclosed methods, the primary cells from the two or more donors may be pancreatic islets, and the pooled cells may be pooled pancreatic islets. At least one of the two or more donors may be a human having a healthy pancreas or a pancreas with a disease or disorder, for example, pancreatitis, pancreatic cancer, or type 1 or type 2 diabetes mellitus (T1DM or T2DM).

The methods disclosed herein may further comprise determining a viability rate of the primary cells from a single donor or at least one of the two or more donors. The primary cells from a single donor or at least one of the two or more donors may have a viability rate less than about 50%, 60%, 70%, 80% or 90%.

The methods disclosed may further comprise determining a viability rate of the primary cells from the single donor or the two or more donors. The primary cells from the single donor or the two or more donors may have a viability rate less than about 50%, 60%, 70%, 80% or 90%.

The disclosed preparation methods may further comprise determining a viability rate of the separated cells from the single donor, or the combined separated cells of the pooled cells. The is separated cells from donor or the combined separated cells may have a viability rate less than about 50%, 60%, 70%, 80% or 90%.

The preparation methods disclosed herein may further comprise determining a viability rate of the pooled enriched cells or the enriched cells from a single donor. The pooled enriched cells may have a viability rate less than about 50%, 60%, 70%, 80% or 90%.

According to the disclosed preparation methods, the primary cells from the single donor or at least one of the two or more donors may have a viability rate less than about 50%, 60%, 70%, 80% or 90%; the primary cells from the single donor or the two or more donors may have a viability rate less than about 50%, 60%, 70%, 80% or 90%; and/or the pooled enriched cells or the enriched cells from the single donor may have a viability rate greater than about 50%, 60%, 70%, 80% or 90%. The primary cells from the single donor or at least one of the two or more donors or from the two or more donors may have a viability rate less than about 50% while the pooled enriched cells or the enriched cells from the single donor may have a viability rate greater than about 50%. The primary cells from the single donor or from at least one of the two or more donors or from the two or more donors may have a viability rate less than about 60% while the pooled enriched cells or the enriched cells from the single donor may have a viability rate greater than about 60%. The primary cells from the single donor or at least one of the two or more donors or from the two or more donors may have a viability rate less than about 70% while the enriched cells from the single donor or the pooled enriched cells may have a viability rate greater than about 70%. The primary cells from the single donor or at least one of the two or more donors or from the two or more donors may have a viability rate less than about 80% while the enriched cells from the single donor or the pooled enriched cells may have a viability rate greater than about 80%. The primary cells from the single donor or at least one of the two or more donors or from the two or more donors may have a viability rate less than about 90% while the enriched cells from the single donor or the pooled enriched cells may have a viability rate greater than about 90%.

According to an embodiment of the preparation method, the pooled enriched cells may have a viability rate greater than the combined separated cells and/or the combined primary cells. The enriched cells from the single donor may have a viability greater than the viability of the primary cells from the single donor. The separated cells from the single donor may have a viability greater than the viability of the primary cells from the single donor. The single donor may be human. The combined separated cells and/or the combined primary cells may have a viability rate greater than the primary cells from at least one of the two or more donors or from the two or more donors. The primary cells from the single donor or at least one of the two or more donors may have a viability rate less than about 50%, 60%, 70%, 80% or 90%. The primary cells from the single donor or the two or more donors may have a viability rate less than about 50%, 60%, 70%, 80% or 90%; the combined primary cells may have a viability rate less than about 50%, 60%, 70%, 80% or 90%; the combined separated cells may have a viability rate less than about 50%, 60%, 70%, 80% or 90%; and/or the pooled enriched cells or the enriched cells from the single donor may have a viability rate greater than about 50%, 60%, 70%, 80% or 90%. The primary cells from the single donor or at least one of the two or more donors or from the two or more donors, the combined human primary cells, and/or the combined separated cells may have a viability rate less than about 50% while the pooled enriched cells or the enriched cells from a single donor may have a viability rate greater than about 50%. The primary cells from the single donor or at least one of the two or more donors or from the two or more donors, the combined human primary cells, and/or the combined separated cells may have a viability rate less than about 60% while the pooled enriched cells or the enriched cells from the single donor may have a viability rate greater than about 60%. The primary cells from the single donor or at least one of the two or more donors or from the two or more donors, the separated cells from the single donor, the combined primary cells, and/or the combined separated cells may have a viability rate less than about 70% while the pooled enriched cells or the enriched cells from the single donor may have a viability rate greater than about 70%. The primary cells from the single donor or at least one of the two or more donors or from the two or more donors, the combined human primary cells, and/or the combined separated cells, or the separated cells from the single donor may have a viability rate less than about 80% while the pooled enriched cells or the enriched cells from the single donor may have a viability rate greater than about 80%. The primary cells from the single donor or at least one of the two or more donors or from the two or more donors, the combined primary cells, and/or the combined separated cells, or the separated cells from the single donor may have a viability rate less than about 90% while the pooled enriched cells or the enriched cells from the single donor may have a viability rate greater than is about 90%.

According to an embodiment of the preparation method, the pooled cells may have a viability rate greater than the combined separated cells and/or the separated cells from at least one of the two or more donors or from the two or more donors. The combined separated cells and/or the separated cells from at least one of the two or more donors or from the two or more donors may have a viability rate greater than the primary cells from at least one of the two or more donors or from the two or more donors. The primary cells from at least one of the two or more donors may have a viability rate less than about 50%, 60%, 70%, 80% or 90%; the primary cells from the two or more donors may have a viability rate less than about 50%, 60%, 70%, 80% or 90%; the separated cells from at least one of the two or more donors may have a viability rate less than about 50%, 60%, 70%, 80% or 90%; the separated cells from the two or more donors may have a viability rate less than about 50%, 60%, 70%, 80% or 90%; the combined separated cells may have a viability rate less than about 50%, 60%, 70%, 80% or 90%; and the pooled cells may have a viability rate greater than about 50%, 60%, 70%, 80% or 90%. The primary cells from at least one of the two or more donors or from the two or more donors, the separated cells from at least one of the two or more donors or from the two or more donors, and/or the combined separated cells may have a viability rate less than about 50% while the pooled cells may have a viability rate greater than about 50%. The primary cells from at least one of the two or more donors or from the two or more donors, the separated cells from at least one of the two or more donors or from the two or more donors, and/or the combined separated cells may have a viability rate less than about 60% while the pooled cells may have a viability rate greater than about 60%. The primary cells from at least one of the two or more donors or from the two or more donors, the separated cells from at least one of the two or more donors or from the two or more donors, and/or the combined separated cells may have a viability rate less than about 70% while the pooled cells may have a viability rate greater than about 70%. The primary cells from at least one of the two or more donors or from the two or more donors, the separated cells from at least one of the two or more donors or from the two or more donors, and/or the combined separated cells may have a viability rate less than about 80% while the pooled cells may have a viability rate greater than about 80%. The primary cells from at least one of the two or more donors or from the two or more donors, the separated cells from at least one of the two or more donors or from the two or more donors, and/or the combined separated cells may have a is viability rate less than about 90% while the pooled cells may have a viability rate greater than about 90%.

According another embodiment of the preparation method, the pooled cells may have a viability rate greater than the combined separated cells and/or the density gradient treated cells from at least one of the two or more donors or from the two or more donors. The combined separated cells and/or the density gradient treated cells from at least one of the two or more donors or from the two or more donors may have a viability rate greater than the primary cells from at least one of the two or more donors or from the two or more donors. The primary cells from at least one of the two or more donors may have a viability rate less than about 50%, 60%, 70%, 80% or 90%; the primary cells from the two or more donors may have a viability rate less than about 50%, 60%, 70%, 80% or 90%; the density gradient treated cells from at least one of the two or more donors may have a viability rate less than about 50%, 60%, 70%, 80% or 90%; the density gradient treated cells from the two or more donors may have a viability rate less than about 50%, 60%, 70%, 80% or 90%; the combined separated cells may have a viability rate less than about 50%, 60%, 70%, 80% or 90%; and the pooled cells may have a viability rate greater than about 50%, 60%, 70%, 80% or 90%. The primary cells from at least one of the two or more donors or from the two or more donors, the density gradient treated cells from at least one of the two or more donors or from the two or more donors, and/or the combined separated cells may have a viability rate less than about 50% while the pooled cells may have a viability rate greater than about 50%. The primary cells from at least one of the two or more donors or from the two or more donors, the density gradient treated cells from at least one of the two or more donors or from the two or more donors, and/or the combined separated cells may have a viability rate less than about 60% while the pooled cells may have a viability rate greater than about 60%. The human primary cells from at least one of the two or more donors or from the two or more donors, the density gradient treated cells from at least one of the two or more donors or from the two or more donors, and/or the combined separated cells may have a viability rate less than about 70% while the pooled cells may have a viability rate greater than about 70%. The primary cells from at least one of the two or more donors or from the two or more donors, the density gradient treated cells from at least one of the two or more donors or from the two or more donors, and/or the combined separated cells may have a viability rate less than about 80% while the pooled cells may have a viability rate greater than about 80%. The primary cells from at least one of the two or more donors or from the two or more donors, the density gradient treated cells from at least one of the two or more donors or from the two or more donors, and/or the combined separated cells may have a viability rate less than about 90% while the pooled cells may have a viability rate greater than about 90%.

The density gradient separations, as described in these methods, separates dying or dead cells from viable cells in a density gradient medium based on the densities of the different types of cells. The density gradient medium may be generated by one or more chemical compounds, biological molecules or combinations thereof. For example, Percoll® provides a density gradient medium suitable for the separation of dead or dying cells from viable cells. Other suitable density gradient media include heavy metal salts, carbohydrates, iodinated compounds and colloidal silica gels. Specific such materials are CsCl, Cs₂O₄, D₂O, Ficoll®, Iodixanol (Visipaque™ or OptiPrep™), Nycodenz®, sorbitol, sucrose, and Percoll®.

According to an embodiment, a solution containing salts is needed to prepare the density gradient. For example, cell culture medium or phosphate buffered saline (PBS) or sodium chloride (NaCl) needs to be added to ensure the solution is isotonic. This also ensures the osmolality of the density gradient solution is correct, and cells will separate out according to their buoyant densities.

According to some embodiments of the disclosed methods, the first density gradient may have a density ranging from about 1.06 g/mL to about 1.25 g/mL. If Percoll® is used, the concentration of the first density separation medium may be from 6% to 50%; or from 6% to 20% (low stringency), or from 20% to 30% (medium stringency), or from 30% to 50% (high stringency).

According to an embodiment of the preparation method, the second density gradient separation may be more stringent than the first density gradient separation. The first density gradient separation may remove a higher percentage of dying or dead cells from the combined human primary cells than the second density gradient separation does from the thawed combined separated cells. The second density gradient separation may use a higher concentration of the density gradient medium, or a stronger centrifugation step, for example, centrifugation at a higher speed and/or for a longer time, than the second density gradient separation.

According to an embodiment, when pooled cells are prepared, the second density gradient (used during pooling) may be more stringent than the first density gradient (used during thaw of the pooled cells). However, according to another embodiment, when the objective is to retain diseased cells and or for single donor applications, the final density gradient may be less stringent than previous gradients, to allow for retention of heterogeneity (as defined by more cell types or cell health differences) in the final culture.

According to some embodiments of the method, the second density gradient may have a density ranging from 1.06 g/mL to 1.25 g/mL If Percoll® is used, the concentration of the second density separation medium may be from 6% to 50% (v/v), or from about 30% to about 50% (v/v) for more stringent applications, or from about 6% to about 20% (v/v) for less stringent applications.

According to an embodiment of the preparation method, the second density gradient separation may be more stringent than the first density gradient separation. The first density gradient separation may remove a higher percentage of dying or dead cells from the human primary cells from the two or more donors than the second density gradient separation does from the thawed combined separated cells. The second density gradient separation may use a higher concentration of the density gradient medium, or a stronger centrifugation step, for example, centrifugation at a higher speed and/or for a longer time, than the second density gradient separation.

According to some embodiments of the method, the first density gradient may have a density ranging from about 1.06 g/mL to about 1.25 g/mL. If Percoll® is used, the concentration of the first density separation medium may be from 6% to 50% (v/v), or from about 6% to about 20%, or from about 20% to about 30%, or from about 30% to about 50%.

According to some embodiments of the method, the second density gradient may have a density ranging from 1.06 g/ml to about 1.25 g/mL. If Percoll® is used, the concentration of the second density separation medium may be from 6% to 50% (v/v), or from 6% to 20%, or from 20% to 30%, or from 30% to 50% 5% to 35% (v/v).

According to some embodiments of the method, the first density gradient may have a density ranging from 1.06 g/mL to 1.25 g/mL. If Percoll® is used, the concentration of the first is density separation medium may be from 6% to 50% (v/v), or from 6% to 20%, or from 20% to 30%, or from 30% to 50% or from 5% to 35% (v/v).

According to some embodiments of the method, the second density gradient may have a density ranging from 1.06 g/ml to about 1.25 g/mL. If Percoll® is used, the concentration of the second density separation medium may be from 6% to 50% (v/v), or from 5% to 45% (v/v), or from 10% to 40% (v/v), or from 15% to 35% (v/v), or from 25% to 30% (v/v).

According to some embodiments of the method, the third density gradient may have a density ranging from 1.01 g/ml to 1.57 g/ml, or from 1.06 g/ml to 1.51 g/ml, or from 11.06 g/mL to 1.25 g/mL. If Percoll® is used, the concentration of the third density separation medium may be from 1% to 50% (v/v), or from 5% to 45% (v/v), or from 10% to 40% (v/v), or from 15% to 35% (v/v), or from 25% to 30% (v/v) or from 6% to 50% (v/v), or from 6% to 20%, or from 20% to 30%, or from 30% to 50% or from 5% to 35% (v/v).

Where the primary cells from the single donor or the two or more donors (where the donor(s) may be human) are frozen, embodiments of the preparation method may further comprise thawing the frozen primary cells from the single donor or each of the two or more donors to generate thawed primary cells from the single donor or from each of the two or more donors. Embodiments of the preparation method may further comprise subjecting the thawed primary cells from the single donor or from each of the two or more donors to a cold recovery step.

A cold recovery step may optionally comprise incubation of cells at about 0-4° C., for example, about 4° C., in a hypothermic preservation solution. The hypothermic preservation solution may comprise one or more nutrients for repair and/or recovery of cells from post-manipulation. The post-manipulation may include thawing or pipetting.

In an embodiment of the method, the primary cells from the two or more donors are combined to generate the combined primary cells, the method may further comprise an optional cold recovery step before removing dying or dead cells from the combined primary cells by the first density separation to generate the combined separated cells. An embodiment of the method may comprise cryopreserving the combined separated cells. An embodiment of the preparation method may further comprise thawing the combined separated cells to generate the thawed is combined separated cells. The method may further comprise subjecting the thawed combined separated cells to a cold recovery step before removing dying or dead cells from the thawed combined separated cells by the second density separation.

In another embodiment of the preparation method the primary cells are from a single donor, the method may further comprise an optional cold recovery step before removing the dying or dead cells from the primary cells by the first gradient density separation to generate separated cells. The method comprises cryopreserving the separated cells. The preparation method may further comprise thawing the separated cells to generate thawed separated cells. The method may further comprise subjecting the thawed separated cells to a cold recovery step before removing the dying or dead cells from the thawed separated cells by the second density separation.

In another embodiment of the preparation method the primary cells are from two or more donors, the method may further comprise an optional cold recovery step for each donor cell separately before removing dying or dead cells from the primary cells by the first density gradient separation to generate the separated cells. The method may comprise combining the separated cells from the two or more donors to generate the combined separated cells and then cryopreserving the cells to generate the cryopreserved combined separated cells. The preparation method may further comprise thawing the cryopreserved combined separated cells to generate the thawed combined separated cells. The method may further comprise subjecting the thawed combined separated cells to a cold recovery step before removing dying or dead cells from the thawed combined separated cells by the second density separation.

In an embodiment of the preparation method, the primary cells are from a single donor, the method may further comprise an optional cold recovery step before removing dying or dead cells from the combined human primary cells by the first density gradient separation to generate the density gradient treated cells. The method may comprise cryopreserving the density gradient treated cells. The preparation method may further comprise thawing the density gradient treated cells to generate the thawed density gradient treated cells. The method may further comprise subjecting the thawed density gradient treated cells to a cold recovery step after combining the cells from two or more donors to generate the combined primary cells and before removing dying or dead cells from the combined primary cells by the second density separation to generate is the combined separated cells. The method may comprise cryopreserving the combined separated cells. The preparation method may further comprise thawing the combined separated cells to generate the thawed combined separated cells. The method may further comprise subjecting the thawed combined separated cells to a cold recovery step before removing the dying or dead cells from the thawed combined separated cells by the third density separation.

According to an embodiment of the preparation method, the pooled enriched cells or the enriched cells from the single donor may maintain a viability rate with a decline less than the primary cells from the single donor or at least one of the two or more donors or from the two or more donors after a predetermined period of time (e.g., about 3, 4, 5 or 6 days or 1, 2, 3, 4, 5, 6, 7, 8, 9 or 10 weeks, or about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour). The pooled enriched cells or the enriched cells from the single donor may maintain a viability rate with a decline less than about 5%, 10%, 15%, 20%, 25%, 30%, 35%, 40%, 45%, 50%, 60%, 70%, 80%, 90%, 95% or 99%, optionally with a deviation no more than about 5, 10, 15, 20 or 25%, after a predetermined period of time (e.g., about 3, 4, 5 or 6 days or 1, 2, 3, 4, 5, 6, 7, 8, 9 or 10 weeks, or about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour); and/or the primary cells from the single donor or at least one of the two or more donors or from the two or more donors may maintain a viability rate with a decline greater than about 5%, 10%, 15%, 20%, 25%, 30%, 35%, 40%, 45%, 50%, 60%, 70%, 80%, 90%, 95% or 99%, optionally with a deviation no more than about 5, 10, 15, 20 or 25%, after a predetermined period of time (e.g., about 3, 4, 5 or 6 days or 1, 2, 3, 4, 5, 6, 7, 8, 9 or 10 weeks, or about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour). The pooled enriched cells may maintain a viability rate with a decline less than a percentage (e.g., about 5%, 10%, 15%, 20%, 25%, 30%, 35%, 40%, 45%, 50%, 60%, 70%, 80%, 90%, 95% or 99%), optionally with a deviation no more than about 5, 10, 15, 20 or 25%, after a predetermined period of time (e.g., about 3, 4, 5 or 6 days or 1, 2, 3, 4, 5, 6, 7, 8, 9 or 10 weeks, or about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour) while the primary cells from at least one of the two or more donors or from the two or more donors or the single donor primary cells may maintain a viability rate with a decline greater than the same percentage, optionally with the same deviation, after the same predetermined period of time.

According to an embedment of the preparation method, the pooled enriched cells or the enriched cells from the single donor may be functionally stable. The pooled enriched cells or the enriched cells from the single donor may maintain a biological function with a less decline than the human primary cells from the single donor or at least one of the two or more donors or from the two or more donors after a predetermined period of time (e.g., about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour). The pooled enriched cells or the enriched cells from the single donor may maintain a biological function with a decline less than about 5%, 10%, 15%, 20%, 25%, 30%, 35%, 40%, 45%, 50%, 60%, 70%, 80%, 90%, 95% or 99%, optionally with a deviation no more than about 5, 10, 15, 20 or 25%, after a predetermined period of time (e.g., about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour, about 3, 4, 5 or 6 days, or about 1, 2, 3, 4, 5, 6, 7, 8, 9 or 10 weeks). The primary cells from the single donor or at least one of the two or more donors or from the two or more donors may maintain a biological function with a decline greater than about 5%, 10%, 15%, 20%, 25%, 30%, 35%, 40%, 45%, 50%, 60%, 70%, 80%, 90%, 95% or 99%, optionally with a deviation no more than about 5, 10, 15, 20 or 25%, after a predetermined period of time (e.g., about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour, about 3, 4, 5 or 6 days, or about 1, 2, 3, 4, 5, 6, 7, 8, 9 or 10 weeks). The pooled cells may maintain a biological function with a decline less than a certain percentage (e.g., about 5%, 10%, 15%, 20%, 25%, 30%, 35%, 40%, 45%, 50%, 60%, 70%, 80%, 90%, 95% or 99%), optionally with a deviation no more than about 5, 10, 15, 20 or 25%, after a predetermined period of time (e.g., about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour, about 3, 4, 5 or 6 days, or about 1, 2, 3, 4, 5, 6, 7, 8, 9 or 10 weeks) while the primary cells from the single donor or at least one of the two or more donors or from the two or more donors may maintain a biological function with a decline greater than the same percentage, optionally with the same deviation, after the same predetermined period of time.

The biological function may be an enzymatic function, or compound uptake via transporter protein. Where the pooled enriched cells or the enriched cells from the single donor are hepatocytes, the biological function may be production of albumin and urea, and cytochrome is P450 enzymatic activity or transporter protein function. When the pooled enriched cells or the enriched cells from the single donor are thyrocytes, the biological function may be proliferation upon stimulation by thyroid stimulating hormone (TSH), TG secretion, or thyroxine (T4) synthesis when the cells are activated with TSH, or by expression of triiodothyronine (T3). When the pooled enriched cells or the enriched cells from the single donor are AT2 lung cells, the biological function may be expression of surfactant protein C. When the pooled enriched cells or the enriched cells from the single donor are pancreatic islets, the biological function may be production of c-peptide and/or insulin, glucagon, somatostatin, pancreatic polypeptide, or other pancreatic hormone.

According to an embodiment of the preparation method, the pooled enriched cells or the enriched cells from the single donor may have culture longevity. At least about 70%, 75%, 80%, 85%, 90%, 95%, 99% or 100% of the pooled enriched cells or the enriched cells from the single donor may remain viable and/or functionally stable for a predetermined period of time (e.g., about 3, 4, 5 or 6 days or 1, 2, 3, 4, 5, 6, 7, 8, 9 or 10 weeks, or about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour); and/or no more than 30%, 40%, 50%, 60%, 70%, 80% or 90% of the primary cells from the two or more donors or the single donor may remain viable and/or functionally stable for a predetermined period of time (e.g., about 3, 4, 5 or 6 days or 1, 2, 3, 4, 5, 6, 7, 8, 9 or 10 weeks, or about 0.01-48, 0.01-24, 0.01-12, 0.01-6, 0.01-4, 0.01-2, 0.01-1, 0.1-48, 0.1-24, 0.1-24, 0.1-12, 0.1-6, 0.1-4, 0.1-2, or 0.1-1 hour).

The pooled enriched cells or the enriched cells from the single donor may have cryo-damaged cells. About 1-40% of the pooled enriched cells or the enriched cells from the single donor may be cryo-damaged cells. The thawed combined separated cells or the separated cells from the single donor may have cryo-damaged cells. About 1-40% of the thawed combined separated cells or the separated cells from the single donor may be cryo-damaged cells. The pooled enriched cells or the cells from the single donor may have a lower percentage of cryo-damaged cells than the thawed combined separated cells or the separated cells from the single donor.

The pooled enriched cells or the enriched cells from the single donor may possess a genetic, for example, epigenetic, feature of a disease. Exemplary genetic features of a disease is include a biological molecule (e.g., gene or protein) or structure associated with the disease. The pooled cells may have an epigenetic modification due to environmental exposure to a chemical compound or a toxicant. The epigenetic modification may be methylation, or histone modification and chromatin remodeling. The epigenetic modification may contribute to a disease or susceptibility risk.

Where at least one of the two or more donors or the single donor is a human suffering from a liver disease or condition, for example, microsteatosis, non-alcoholic steatohepatitis (NASH), non-alcoholic fatty liver disease (NAFLD) or hepatitis (e.g., A, B, C, D and E), the pooled cells may possess a biomarker associated with the liver disease or condition. Hepatocytes from a NASH disease liver may express biomarkers such as cytokeratin 18 (CK18).

Where the two or more donors or the single donor is a human suffering from a thyroid disease or disorder, for example, hyperthyroidism, hypothyroidism, Hashimoto's thyroiditis, Graves' disease, goiter, thyroid nodules, thyroid tumors, or thyroid cancer, the pooled enriched cells or the enriched cells from the single donor may possess a biomarker associated with the thyroid disease or condition. The pooled enriched cells or the enriched cells from the single donor may be positive for a biomarker selected from the group consisting of cytokeratin 7 (CK7), thyroglobulin (TG), epithelial cellular adhesion molecule (EpCAM), thyroid stimulating hormone (TSH) receptor and thyroperoxidase (TPO) receptor.

Where the two or more donors or the single donor is a human suffering from a lung disease or disorder, for example, idiopathic pulmonary fibrosis (IPF) which may include a pro-fibrotic phenotype, lung adenocarcinoma (LUAD), acute lung injury (ALI) (e.g., pneumonia, inhalation of toxic substances, fat embolism, viral infection of the lungs), acute respiratory distress syndrome (ARDS), hyperplasia, or chronic obstructive pulmonary disease (COPD), the pooled enriched cells or the enriched cells from the single donor may possess a biomarker associated with the lung disease or condition, such as surfactant protein c.

Where the two or more donors or the single donor is a human suffering from a pancreatic disease or disorder, for example, pancreatitis, pancreatic cancer, or type 1 or type 2 diabetes mellitus (T1DM or T2DM), the pooled enriched cells or the enriched cells from the single donor may possess a biomarker associated with the pancreatic disease or condition, such as insulin, glucagon, somatostatin, and pancreatic polypeptide.

For each preparation method of the present invention, a product comprising the pooled enriched cells or the enriched cells from the single donor prepared according to the preparation method is provided. The pooled enriched cells or the enriched cells from the single donor may be a mixture of single cells and aggregates of two or more cells.

Where the pooled enriched cells are prepared from human primary cells from two or more donors in a target geographical region, the pooled enriched cells may be used to recapitulate the population heterogeneity of the target geographical region by maintaining demographic ratios of the targeted geographical region in the donors selected for a pooled enriched cell product. Ethnicity may also be reflected; for example, Latino can be used indicating that these donors are from Latin America or the Caribbean.

The pooled enriched cells or the enriched cells from the single donor in the product may be pooled enriched hepatocytes or enriched hepatocytes from a single donor. The pooled enriched hepatocytes or the enriched hepatocytes from a single donor may be in suspension. The pooled enriched hepatocytes or the enriched hepatocytes from a single donor may be plated on a surface. At least about 50%, 60%, 70%, 80% or 90% of the plated pooled enriched hepatocytes or the plated enriched hepatocytes from a single donor may be in one or more hepatocyte clusters on feeder cells. The feeder cells may be endothelial cells and fibroblasts, and may be attached to the surface.

The product may further comprise a culture system. The culture system may be selected from the group consisting of a microphysiological system (MPS), a self-assembled organoid, a bioprinted tissue, a micropatterned culture, a multi-cellular spheroid, a cell/hydrogel hybrid model, and a cell/device hybrid model. An MPS may be an interconnected set of two- or three-dimensional cellular constructs, which are frequently referred to as organs-on-chips or in vitro organ constructs. A self-assembled organoid may be a three-dimensional, self-assembled cell aggregate that self-patterns based on cell differentiation and modulation by morphogenic agents, mimicking a natural organ and its complex processes. A bioprinted tissue may be a replica of a natural tissue that is created through 3D printing of cells in their naturally occurring patterns using a bioink, a substance supportive to the cells and overall formation of the tissue. A micropatterned culture may be cells cultured on a patterned substrate with adherent domains of is predefined geometric features. A multi-cellular spheroid may be a self-assembled, scaffold-free multi-cellular aggregate that may be composed of multiple cell types. A cell/hydrogel hybrid model may be cells cultured with a single or combination of natural and synthetic polymers that are more liquid like at lower temperatures and become more gel like at higher temperatures, and mimic extracellular matrices to recapitulate the native cell environment. A cell/device hybrid model may be cells, a single or multiple types, cultured in a device that allows for more physiologically relevant conditions such as the flow of fluids, allowing for a constant nutrient supply, waste removal, and mimicking sheer stress.

A method of testing a pharmaceutical substance is provided. The method comprises administering a pharmaceutical substance to pooled enriched cells or enriched cells from a single donor in an amount effective to change a property of the pooled enriched cells or the enriched cells from a single donor. The pooled enriched cells or the enriched cells from a single donor are prepared according to a method of the present invention or in the product of the present invention. The pooled enriched cells may be pooled enriched hepatocytes, pooled enriched thyrocytes, pooled enriched AT2 lung cells, or pooled enriched pancreatic islets. The enriched cells from a single donor may be enriched hepatocytes, enriched thyrocytes, enriched AT2 lung cells, or enriched pancreatic islets. The pharmaceutical substance may be a chemical compound, a biological molecule or a combination thereof. The pharmaceutical substance may comprise at least one of small molecules, lipid nanoparticles, antibodies, bacteria or derivatives thereof, live viruses (e.g., hepatitis B or C), viral vectors, oligonucleotides and cells. Examples of the bacteria or derivatives thereof include liposomes, plasmid vectors, and cell-derived materials including microvesicles, exosomes, lysates, conditioned media, and secreted proteins.

A method of testing drug metabolism is provided. The method comprises administering an effective amount of a drug to pooled enriched cells or enriched cells from a single donor. The pooled enriched cells or enriched cells from a single donor are prepared according to a method of the present invention or in the product of the present invention. The pooled enriched cells may be pooled enriched hepatocytes, pooled enriched thyrocytes, pooled enriched AT2 lung cells, or pooled enriched pancreatic islets. The enriched cells may be enriched hepatocytes, enriched thyrocytes, enriched AT2 lung cells, or enriched pancreatic islets. The drug may be a chemical compound, a biological molecule or a combination thereof. The enriched pooled cells or the is enriched calls from a single donor may be in a culture medium. The term “drug metabolism” as used herein refers to conversion or clearance of a drug. A drug metabolite may be generated. The method may further comprise determining the amount of the drug or the drug metabolite in the pooled enriched cells, the enriched cells from a single donor or the culture medium.

A method of testing drug transport is provided. The method comprises administering an effective amount of a drug to pooled enriched cells or enriched cells from a single donor. The pooled enriched cells or the enriched cells from a single donor are prepared according to a method of the present invention or in a product of the present invention. The pooled enriched cells may be pooled enriched hepatocytes, pooled enriched thyrocytes, pooled enriched AT2 lung cells, or pooled enriched pancreatic islets. The enriched cells from a single donor may be enriched hepatocytes, enriched thyrocytes, enriched AT2 lung cells, or enriched pancreatic islets from a single donor. The drug may be a chemical compound, a biological molecule or a combination thereof. The method may further comprise determining the cellular uptake and distribution of the drug in the pooled enriched cells or the enriched cells from a single donor.

A method of testing drug toxicity is provided. The method comprises administering an effective amount of a drug to pooled enriched cells or enriched cells from a single donor. The pooled enriched cells are prepared according to a method of the present invention or in a product of the present invention. The pooled enriched cells may be pooled enriched hepatocytes, pooled enriched thyrocytes, pooled enriched AT2 lung cells, or pooled enriched pancreatic islets. The enriched cells from a single donor may be enriched hepatocytes, enriched thyrocytes, enriched AT2 lung cells, or enriched pancreatic islets from a single donor. The drug may be a chemical compound, a biological molecule or a combination thereof. The method may further comprise detecting a toxic event. The detection of a toxic event may be evidenced by a reduced percentage of remaining viable cells in the pooled enriched cells or the enriched cells from a single donor.

A method of preparing a hepatitis B virus (HBV) infected hepatocyte culture model is provided. The method comprises inoculating pooled enriched hepatocytes or enriched cells from a single donor with hepatitis B virus (HBV) and incubating the infected pooled enriched hepatocytes or enriched hepatocytes from a single donor in a culture medium for at least 7, 14 or 21 days, for example, 14 days. The HBV infected hepatocyte culture model is prepared. The pooled enriched hepatocytes or enriched hepatocytes from a single donor are prepared according to a method of the present invention or in a product of the present invention. The pooled enriched hepatocytes may be neonatal or juvenile hepatocytes. The enriched hepatocytes from a single donor may be neonatal or juvenile hepatocytes. The pooled enriched hepatocytes or the enriched hepatocytes from a single donor may be plated hepatocytes. The plated hepatocytes (pooled or from a single donor) may be in co-culture with feeder cells and/or macrophages, for example, liver-derived macrophages (e.g., Kupffer cells). The method may further compromise determining transcription or expression level of a liver-specific bile acid transporter, for example, sodium taurocholate cotransporting polypeptide (NTCP), of the pooled hepatocytes, selecting a batch of pooled enriched hepatocytes or batch of enriched hepatocytes from a single donor with a desirable transcription or expression level of NTCP, plating the selected batch of pooled hepatocytes or selected batch of hepatocytes from a single donor before inoculating the plated hepatocytes with hepatitis B virus (HBV). An overlay of protein matrix may not be required to increase the virus inoculation efficiency.

The term “about” as used herein when referring to a measurable value such as an amount, a percentage, and the like, is meant to encompass variations of ±20% or ±10%, more preferably ±5%, even more preferably ±1%, and still more preferably ±0.1% from the specified value, as such variations are appropriate.

### EXAMPLES

### Materials and Methods

### Density Gradient Steps

Isolation of Hepatocytes was done with 20-30% (v/v) Percoll®*.

Pooling of Hepatocytes was done with 27% (v/v) Percoll®†.

Post Thaw of Pooled Hepatocytes was done with 27-35% (v/v) Percoll®.

*Percoll® percentage varies based on donor disease state and condition of hepatocytes.

†Percoll® percentages listed are those predominantly used for hepatocytes from “healthy” donors. The Percoll® percentage may be varied for the pooling step if necessary to clean up certain lots more than others when handled separately. In addition, these percentages may change, likely to be lower, if hepatocytes are pooled from donors with a disease state. The concentration (% v/v) percentages could fall between 6-50% (v/v) Percoll® for hepatocytes from is donors with a disease state, depending on the desired final cell populations and on how the hepatocytes may be affected by the particular disease state. In these cases, the centrifugation speed and duration may also need to be modified.

Example process for pooling already cryopreserved hepatocytes is as follows.
1. Thaw vials from donor lots.
2. Mix all lots in ColdPreserv (hyperthermic preservation solution available from VitroPreP LLC) and DNase (enzymes that break down DNA by breaking the phosphodiester bonds in DNA's backbone). Mixing and thawing was done for two hours at 4° C. in a spinner flask at 70 RPM.
3. Mix cells with Percoll® to a total concentration of Percoll® of 27% (v/v).
4. Centrifuge the cells and Percoll® at 200 G for 10 minutes at 4° C.
5. Resuspend the cells in cold HHPM (completed human hepatocyte plating medium form LifeNet Health).
6. Count the cells to determine viability.
7. Cryopreserve the suspension in NG5A CryoPreserve (a biopreservation medium used for freezing and shipping cells, available from VitroPrep LLC).

### Post-Thaw Processing of Cryopreserved Pooled Hepatocytes

1. Thaw vial of pooled cells prepared as above. The cells are thawed in 27% (v/v) Percoll®.
2. Centrifuge the thawed pooled cells in the 27% (v/v) Percoll®. Centrifuging was done at 100 G for 8 minutes at room temperature (20-25° C.).
3. Resuspend the centrifuged cells in HHPM.
4. Count the cells to determine percent viability.

### TruVivo® Cell System

The TruVivo® cell system was set up as previously described, including the medias, cell types, and expected functional performance. PHHs (primary human hepatocytes) were thawed in medium containing either low (diluted 4-fold) or high (standard 27% v/v) Percoll®. 300,000 PHHs and 50,000 human FCs (feeder cells) were seeded per well in a 24-well plate. All methods were performed in accordance with the guidelines and regulations of LifeNet Health's ethics committee. Informed consent was obtained for all donor tissue for research purposes by LifeNet Health. PHHs have been designated as normal or diseased based on a histopathologic assessment of the tissue of origin by a board-certified pathologist, using the standard NASH CRN Scoring System. Tissues with a NAS score of ≥4 or donors diagnosed with T2DM were designated “Diseased” while those with a NAS score of ≤3 were categorized as “Normal” (Table 1).

TABLE 1
Donor Characteristics. Donor characteristics including age, sex, race, and BMI.
NAS score consists of Steatosis Score, Lobular Inflammation Score, and Hepatocyte
Ballooning Score. The Fibrosis stage of each donor lot is listed.

| | | | | | | | Lobular | Hepatocyte | |
|---|---|---|---|---|---|---|---|---|---|
| | | | | | NAS | Steatosis | Inflammation | Ballooning | Fibrosis |
| Donor | Age | Sex | Race | BMI | Score | Score | Score | Score | Score |
| 161117 | 41 | F | Cauc. | 30 | 0 | 0 | 0 | 0 | 0 |
| 2118143 | 44 | M | Cauc. | 30.1 | 3 | 1 | 2 | 0 | 2 |
| 2116167 | 51 | M | Cauc. | 29.8 | 4 | 1 | 1 | 2 | 1 |
| 16096 | 73 | F | Cauc. | 30 | 4 | 2 | 1 | 1 | 0 |
| 1811122 | 28 | F | Hisp. | 34 | 5 | 3 | 0 | 2 | 1A, |
| | | | | | | | | | focal |
| 2113766 | 56 | M | Cauc. | 23.9 | 1 | 0 | 1 | 0 | 22 |
| 2118545 | 59 | M | Cauc. | 28.6 | 2 | 1 | 0 | 1 | 0 |

### Morphological Assessment and PHH Attachment

To morphologically assess the cells, they were imaged on the designated days using a BX41 microscope (Olympus, Tokyo, Japan) or Zeiss Observer.Z1 fluorescent microscope (Zeiss, Dublin, CA). The previously described method was used to determine PHH attachment.

### Albumin and Urea Assays

Supernatant was collected on the indicated days for measurement of albumin and urea. Three wells or greater were designated for each condition unless otherwise stated. Samples were run in duplicate. The concentration of albumin was determined using an ELISA assay (Abcam, Cambridge, MA) and performed according to the manufacturer's instructions. Urea was measured by a colorimetric kit (Stanbio, Boerne, TX) and performed according to the manufacturer's instructions.

### Basal CYP3A4 Activity Assay

The P450-Glo Assay kit was used to detect baseline CYP3A4 enzyme activity (Promega, Madison, WI). After 24 hours, the medium was removed, and the cells were washed with DMEM (no phenol red) (Thermo Fisher, Waltham, MA). Cyp-Luciferin-IPA stock was then added and incubated at 37° C. for 30 minutes. The supernatant was then collected, and the assay was performed according to the manufacturer's instructions.

### Gene Expression

Cells were lysed using RLT buffer (Qiagen, Germantown, MD). RNA was then isolated using the RNeasy kit (Qiagen) as per the manufacturer's instructions. cDNA was prepared using the PrimeScript RT reagent kit (Takara Bio, Shiga, Japan) in a 30 μL volume reaction containing 6 μL 5× PrimeScript RT Master Mix, 20 μL RNA, and 4 μL ddH₂O. qRT-PCR reactions contained 10 μL QuantiNova 2×SYBR Green Master Mix (Qiagen), 2 μL ROX reference dye (1:10 dilution; Qiagen), 2 μL designated primer set (10 pM), 1 μL cDNA, and 5 μL ddH₂O for a final volume of 20 μL. Primer sequences used for Glyceraldehyde 3-Phosphate Dehydrogenase (GAPDH), G6PC, and PCK1 (Thermo Fisher) are shown in Table 2. PCR amplification was done on a QuantStudio™ 7 Flex Real-time PCR System (Thermo Fisher) using the following program: Step 1: 02:00 minutes at 95° C.; Step 2) 00:05 seconds at 95° C.; Step 3) 00:10 seconds at 60° C. Repeat steps 2 and 3 for 40 cycles. Data was analyzed with QuantStudio™ 7 Flex Real-Time PCR System software (Thermo Fisher) and Microsoft Excel. Gene expression was normalized to the housekeeping gene GAPDH and analyzed using the 2^{-ΔΔCT} method. It is acknowledged that the use of GAPDH as the housekeeping gene could lead to the exclusion of certain data sets. Data is presented as fold change relative to PHHs enriched in high Percoll®.

TABLE 2
Primer sequences for RT-PCR. Primer sequences
are listed for Glyceraldehyde-3-phosphate
dehydrogenase (GAPDH), Glucose-6-Phosphate
Catalytic subunit (G6PC), Phosphoenolpyruvate
Carboxykinase (PCK1).
GAPDH
Forward: 5′-GGTCACCAGGGCTGCTTTTA-3′
Reverse: 5′-GGATCTCGCTCCTGGAAGATG-3′
G6PC
Forward: 5′-TCATCTTGGTGTCCGTGATCG-3′
Reverse: 5′-TTTATCAGGGGCACGGAAGTG-3′
PCK1
Forward: 5′-ACTCGAGGTTCTGCACCCCT-3′
Reverse: 5′-AGGCAGCATCAATGATGGG-3′

### Immunofluorescence and Quantitation

Cells were fixed with a Fixation Solution (eBioscience, San Diego, CA) for 30 minutes (mins) at 4° C. They were then washed two times with 1× Permeabilization (eBioscience), and primary antibody was added at 4° C. overnight. The following antibodies were used: 1) CD68 at 1:100 (Abcam, ab955); 2) CD163 at 1:100 (Abcam, ab87099); 3) CK18 at 1:1000 (Abcam, ab24561); 4) TGF-β at 1:100 (Abcam, ab92486). Cells were washed twice, and secondary goat anti-mouse IgG Alexa Fluor 488 conjugated antibody (Thermo Fisher) or secondary goat anti-rabbit IgG Alexa Fluor 555 conjugated antibody (Thermo Fisher) was added at a 1:500 dilution for 30 mins at 4° C. Cells were then washed twice, and Fluoromount-G mounting medium with DAPI (Invitrogen, Waltham, MA) was added for 20 mins at room temperature. Images were taken using a Zeiss Observer.Z1 fluorescent microscope.

To quantitate the signal, five images were taken from a pre-determined location for each sample well. Each well was focused in the 5× objective, followed by capturing images at the 1× objective. Exposure for DAPI, dsRed, and GFP was set at 150 ms, 1000 ms, and 2000 ms, respectively. Images were processed in ZEN software to remove fluorescent background by adjusting image grey values. All fluorescent channels were set using the same grey values determined for their specific filter. Individual and merged images were exported and opened in Image J. Individual channels were measured for fluorescent intensity using the “measure” function in ImageJ set to record integrated density. Quantitation of signal is represented in Relative Fluorescent Units (RFUs).

### Nile Red Staining

Cells were washed with 1×DPBS (−Ca⁺⁺/−Mg⁺⁺) (Thermo Fisher) three times, and then Nile Red (Abcam) was added at a 1:500 dilution. After 15 mins at 37° C., cells were washed twice with 1×DPBS (−Ca⁺⁺/−Mg⁺⁺) and then imaged on an EVOS FL cell imaging system (Thermo Fisher) using a 10× or 20× objective. Nile Red staining was quantified by determining the densitometric fluorescence value (red channel) using Image J.

### Calculating PHH Attachment and Statistical Analysis

Images shown are from representative donor lots. For albumin, urea, and CYP3A4 measurements, values were normalized to the determined number of attached PHHs as described above. Significance was calculated in MiniTab (State College, PA) using either Student's t-test or one-way ANOVA with Tukey post hoc testing to determine statistical significance with 95% confidence and *p<0.05 for statistical significance. The Student's t-test was used to calculate significance when comparing two groups, while the ANOVA with Tukey post hoc testing was used when comparing 3 groups or more.

Characterization of T2DM Donors after Enrichment in Low and High Percoll®

Two T2DM donors, 2113766 and 2118545, were enriched in low and high Percoll® after thawing (FIG. 1). Although these donors did not have a NAS score high enough to classify them as diseased, T2DM is strongly correlated with NAFLD. There were distinct morphological is differences on days 7 and 14 between these donor lots after enrichment in both thawing medias (FIG. 1a). Donor lot 2118545 had the normal cuboidal cell shape and formed hepatocyte colonies. Irregularly shaped hepatocytes with large vacuoles were seen in donor lot 2113766. Although there were differences in attachment after the use of different thawing medias for donor lot 2113766 (105,856±64,145 vs 49,472±15,970 PHHs), they were not significant (FIG. 1b). Enrichment in the low Percoll® thawing medium resulted in significantly lower attachment for donor lot 2118545 compared to the high Percoll® thawing medium (126,304±25,583 vs 166,848±14,063 PHHs).

Differences in functionality were measured within the donor lots after enrichment using either thawing medium. For donor lot 2113766, significant differences were seen in albumin levels (low: 15.0±1.4 vs high: 22.6±2.0 μg/10⁶ PHHs/day) (FIG. 1c) but not in urea levels (low: 10.1±1.8 vs high: 8.4±1.4 μg/10⁶ PHHs/day) (FIG. 1d) on day 14. Baseline CYP3A4 levels on day 14 were significantly lower in the PHHs enriched in the low Percoll® (6.4±0.4 nm/10⁶ PHHs/day) versus those enriched in high Percoll® (11.6±0.7 nm/10⁶ PHHs/day) (FIG. 1e). Significantly higher levels of albumin were seen in donor lot 2118545 after enrichment in low Percoll® compared to high Percoll® (low: 36.5±1.0 vs high: 32.6±1.7 μg/10⁶ PHHs/day). There were no significant differences seen in urea levels (low: 43.3±4.7 vs high: 49.5±1.8 μg/10⁶ PHHs/day) and baseline CYP3A4 activity (low: 9.1±1.0 vs high: 9.6±0.4 nm/10⁶ PHHs/day) after post-thaw enrichment in either low or high Percoll®.

Donor lot 2113766 has a fibrosis score 2 compared to fibrosis score 0 for donor lot 2118545. The fibrotic markers, CK18 and TGF-β, were examined to determine differences in expression between the cell populations enriched in either low or high Percoll® (FIG. 2). Representative images of CK18 (FIG. 2a) and TGF-β (FIG. 2b) on day 14 show more intense staining for both markers in donor lot 2113766 after enrichment in either low or high Percoll®. After quantitating the fluorescent signal, expression was significantly higher in PHHs enriched in high Percoll® for CK18 (low: 953.4±133.8 vs high: 1,513±67.5 RFUs/PHHs) (FIG. 2c) and TGF-β (low: 82.7±29.3 vs high: 214.2±79 RFUs/PHHs) (FIG. 2d) for donor lot 2113766. Fibrotic marker expression for CK18 was significantly higher in the low Percoll® enriched PHHs (747±51.3 RFUs/PHHs) compared to the PHHs enriched in high Percoll® (559±17.4 RFUs/PHHs) for donor lot 2118545. Although TGF-β was higher in the low enriched Percoll® PHHs (89.2±31.4 RFUs/PHHs), it was not significant compared to the PHHs enriched in the high Percoll® (58.4±11.6 RFUs/PHHs).

Lipogenesis on day 14 from these two lots was examined using Nile Red staining between PHHs that were enriched in either low or high Percoll® (FIG. 2e). Following enrichment with either low or high Percoll®, lot 2113766 appeared to have more intense staining throughout the PHHs as compared to lot 2118545, where staining was less intense and concentrated on the periphery. When the fluorescence was quantitated, PHHs from lot 2113766 enriched in high Percoll® had significantly higher fluorescence (346.4±61.1 RFUs) compared to those enriched in low Percoll® (low: 221.1±27.1 RFUs) (FIG. 2f). The opposite was measured in PHH lot 2118545. Significantly higher fluorescent signal was measured from PHHs enriched in the low Percoll® (187.3±11.6 RFUs) than those enriched in the high Percoll® (142.3±13.1 RFUs).

Characterization of Diseased Steatotic Donors after Enrichment in Low and High Percoll®

Two diseased donor lots, 1811122 and 16096, and one normal donor lot, 16117, were thawed and then enriched using either low or high Percoll® (FIG. 3). Donor lot 1811122 has a steatosis score of 3, while a score of 2 was given to donor lot 16096. Normal donor lot 16117 has no steatosis score. There were morphological differences on days 7 and 14 in the diseased donor lots that were not seen in the normal donor lot after enrichment in either the low or high Percoll® (FIG. 3a). No significant differences in attachment were determined for donor lots 1811122 (low: 9,120±3,194 vs high: 17,120±8,102 PHHs), 16096 (low: 12,352±2,838 vs high: 15,648±4,212 PHHs), and 16117 (low: 107,360±4,302 vs high: 110,656±13,541 PHHs) after enrichment using either thawing medium (FIG. 3b). However, PHHs from normal donor lot 16117 had significantly higher attachment after enrichment in either low or high Percoll® compared to the PHHs enriched in either low or high Percoll® from the diseased donor lots.

When steatosis was examined, the diseased PHHs appeared to have increased lipogenesis compared to the normal lot when stained using Nile Red on day 14 (FIG. 3c). The diseased lots enriched in the low Percoll® appeared to have more intense staining throughout the hepatocytes is and therefore more lipid accumulation compared to the diseased PHHs enriched in the high Percoll®. Lipid accumulation was seen along the periphery in the normal donor lot and was less intense compared to the diseased lots. When Nile Red staining was quantified, the diseased lots had significantly higher fluorescence after enrichment in the low Percoll® (1811122: 3,312±580 RFUs; 16096: 3,288±553 RFUs) compared to those enriched in the high Percoll® (1811122: 2,035±352 RFUs; 16096: 2,684±292 RFUs) (FIG. 3d). There was no significant difference between the fluorescent signal from the normal donor lot 161117 after enrichment in either the low (395.6±35 RFUs) or high Percoll® (436.7±60.1 RFUs).

The functionality of the diseased donor lots and the normal donor lot was determined, including albumin and urea levels and CYP3A4 activity on day 14 (FIG. 4). There were no significant differences in albumin levels after enrichment in low and high Percoll® for diseased lot 1811122 (low: 35.2±1.6 vs high: 35.6±2.2 μg/10⁶ PHHs/day) and normal lot 16117 (low: 26.9±0.9 vs high: 29.9±2.1 μg/10⁶ PHHs/day) (FIG. 4a). Significantly higher levels of albumin were measured from diseased donor lot 16096 after enrichment in high Percoll® (42.5±1.3 g/10⁶ PHHs/day) compared to low Percoll® (32.8±1.7 μg/10⁶ PHHs/day). PHHs from this donor lot enriched in high Percoll® also had significantly higher levels of urea than those enriched in low Percoll® (low: 65.7±1.7 vs high: 88.5±4.1 μg/10⁶ PHHs/day) (FIG. 4b). There were no significant differences in urea levels in donor lots 18111122 and 16117 after enrichment in either low (1811122: 39.3±1.5 μg/10⁶ PHHs/day; 16117: 72.8±0.6 μg/10⁶ PHHs/day) or high Percoll® (1811122: 37.8±6.8 μg/10⁶ PHHs/day; 16117: 74.8±1.5 ag/10⁶ PHHs/day).

In addition to albumin and urea, baseline CYP3A4 activity was measured for all 3 donor lots and was significantly higher in PHHs enriched in high Percoll® (FIG. 4c). Diseased donor lot 16096 had the highest activity (low: 28.9±2.7 vs high: 43.3±1.7 nm/10⁶ PHHs/min) while the other diseased donor lot 1811122 had the lowest activity (low: 2.7±0.7 vs high: 5.0±0.5 nm/10⁶ PHHs/min). Donor lot 16117 had significantly lower CYP activity in PHHs enriched in low Percoll® (21.2±1.5 nm/10⁶ PHHs/min) compared to those PHHs enriched in high Percoll® (23.4±1.1 nm/10⁶ PHHs/min).

Gene expression of Glucose-6-Phosphatase Catalytic Subunit (G6PC) and Phosphoenolpyruvate Carboxykinase 1 (PCK1) were measured on day 14 in these donor lots after enrichment in low and high Percoll® (FIG. 4d). There was a significant fold-change is decrease in G6PC gene expression after enrichment in the low Percoll® for both diseased lots 1811122 (2.7±0.2) and 16096 (2.2±0.5) when normalized to the PHHs enriched in the high Percoll® (1811122: 1.3±0.3; 16096: 0.9±0.1). No change in expression of this gene was measured after enrichment in low Percoll® (0.8±0.1) when normalized to PHHs enriched in high Percoll® (0.9±0.2) for normal donor lot 16117. For PCK1 gene expression, PHHs from diseased lot 16096 enriched in low Percoll® (1.4±0.2) had significantly decreased fold-change expression when normalized to PHHs enriched in high Percoll® (1.0±0.1). Although PHHs from diseased lot 1811122 enriched in low Percoll® had decreased gene expression (4.9±1.4) after normalization to high Percoll® enriched PHHs (2.5±1.7), it was not significant. No differences in PCK1 gene expression were seen in PHHs enriched in low Percoll® (0.5±0.0) when normalized to PHHs enriched in high Percoll® (0.8±0.3) from normal donor lot 16117.

Differences in Expression of Fibrotic Markers after Enrichment of PHHs in Low and High Percoll®

Two additional donor lots, 2118143 and 2116167, with fibrosis scores of 2 and 1 and a normal donor lot, 16117, with a fibrosis score of 0 were enriched in low and high Percoll® after thawing (FIG. 5). The PHHs in the fibrotic donor lots enriched in the low Percoll® appeared to have a different morphology on days 7 and 14 compared to the PHHs enriched in the high Percoll® (FIG. 5a). These high Percoll® enriched PHHs were cuboidal in shape, multinucleated, and formed hepatic colonies with well-defined borders by day 7. This morphology was maintained throughout the 14-day culture period. However, the low Percoll® enriched PHHs appeared flat with less distinct colony formation and borders. Differences in attachment on day 14 were measured after enrichment using the different thawing medias (FIG. 5b). The PHHs enriched in the low Percoll® attached significantly less compared to the high Percoll® enriched PHHs for the diseased lots 2118143 (low: 55,856±6,624 vs high: 110,092±11,079 PHHs) and 2116167 (low: 70,502±12,866 vs high: 110,783±11,288 PHHs). There was no significant difference in attachment for the normal donor lot 16117 between low (108,617±15,658 PHHs) versus high (108,184±13,298 PHHs) Percoll® enrichment.

Differences in lipogenesis on day 14 were determined after enrichment in low and high Percoll® for lots 2118143 and 2116167 (FIG. 5c). Similar Nile Red staining patterns were seen in both lots with lipids accumulating along the cell periphery and within the PHHs enriched using the low Percoll®. PHHs enriched in high Percoll® appeared to have lipids located mostly along the periphery and little to no lipid accumulation within the cells. Quantification of Nile Red fluorescent signal was determined (FIG. 5d). There was significantly higher fluorescent signal measured in the PHHs enriched in the low Percoll® (2118143: 270.0±28.1 RFUs; 2116167: 270.0±30.3 RFUs) compared to those enriched in the high Percoll® (2118143: 195.9±16.0 RFUs; 2116167: 198.8±6.0 RFUs).

Hepatocyte function was determined for each lot after being enriched in either the low or high Percoll® (FIG. 6). There were significantly lower levels of albumin for lots 2118143 and 2116167 after enrichment in low (2118143: 13.3±1.4 μg/10⁶ PHHs/day; 2116167: 11.0±1.9 g/10⁶ PHHs/day) versus high (2118143: 25.1±1.5 μg/10⁶ PHHs/day; 2116167: 22.4±1.7 g/10⁶ PHHs/day) Percoll® (FIG. 6a). The level of urea was also significantly higher in the PHHs enriched using the high Percoll® for both lots (2118143: 53.9±2.5 μg/10⁶ PHHs/day; 2116167: 54.7±1.7 μg/10⁶ PHHs/day) compared to PHHs enriched in low Percoll® (2118143: 40.2±3.7 μg/10⁶ PHHs/day; 2116167: 36.5±8.7 μg/10⁶ PHHs/day) (FIG. 6b).

Expression of the fibrosis marker CK18 on days 7 and 14 was determined in each lot after enrichment using the two different Percoll® medias (FIG. 6c). CK18 staining appeared to be more intense in the diseased lots 2118143 and 2116167 at both time points after enrichment in the low Percoll®. Quantification of this staining on day 7 showed significantly higher levels of fluorescence in the PHHs enriched in the low Percoll® for lots 2118143 (2,812.6±235.3 RFUs) and 2116167 (2,115.9±234.2 RFUs) compared to those enriched in the high Percoll® (2118143: 1,479.2±183.8 RFUs; 2116167: 1,373.1±117.9 RFUs) (FIG. 6d). Significantly higher staining was also determined on day 14 for both lots when enriched in low Percoll® (2118143: 2,279.7±257.1 RFUs; 2116167: 2,009.3±286.9 RFUs) compared to high Percoll® (2118143: 1,324.4±118.4; 2116167: 1,390.8±63.9 RFUs). For lot 2118143, a significant decrease in CK18 staining was measured on day 14 compared to day 7 from the PHHs enriched in the low Percoll®. This decrease was not seen in the other lots. There were no significant is differences seen after enrichment in either low (day 7: 1,391.9±174.5 RFUs; day 14: 1,544.4±109.9 RFUs) or high Percoll® (day 7: 1,367.5±123.5 RFUs; day 14: 1,518.1±125.7 RFUs) for the normal lot 16117.

Inflammation in Fibrotic PHHs after Enrichment in Low and High Percoll®

When staining for the macrophage marker CD68 was performed in the fibrotic donor lots 2118143 and 2116167, there was light staining on day 7 in PHHs enriched in low Percoll® compared to those high Percoll® enriched PHHs (Supp. FIG. 1). More intense staining was seen on day 14 in the fibrotic lots compared to day 7 (FIG. 7). The diseased lot 2116167 appeared to have the most intense CD68 staining on day 14 from PHHs enriched in either Percoll® thawing medium (FIG. 7a). There appeared to be little to no change in staining intensity on days 7 (Supp. FIG. 1a) and 14 in the normal donor lot 16117 from PHHs enriched in either low or high Percoll®. When the fluorescent signal was quantified, it was higher in the PHHs enriched in the low Percoll® for both fibrotic lots 2118143 (258.1±85.8 RFUs) and 2116167 (1,146.4±216.6 RFUs) compared to PHHs enriched in the high Percoll® (2118143: 151.9±35.9 RFUs; 2116167: 189.9±48.2 RFUs) on day 14 (FIG. 7b). However, only diseased lot 2116167 had a significantly higher signal. The signal on day 7 was also higher in the fibrotic donor PHHs enriched in the low Percoll® (2118143: 46.1±8.4 RFUs; 2116167: 73.0±22.3 RFUs) compared to those PHHs enriched in the high Percoll® (2118143: 17.8±2.3 RFUs; 2116167: 20.5±5.6 RFUs) (Supp. FIG. 1b). No significant differences were determined in the normal lot 16117 on days 7 (low 13.6±8.4 vs high 12.2±2.3 RFUs) and 14 (low 16.8±9.3 vs high 24.1±7.5 RFUs) using either Percoll® thawing medium.

A similar pattern of expression was seen for the macrophage marker CD163 in the fibrotic versus normal donor lots. Little to no fluorescence was seen in the PHHs on day 7 from any of the 3 lots after enrichment in either low or high Percoll® (Supp. FIG. 1c). There appeared to be an increase in fluorescent intensity on day 14 in the fibrotic lots from the PHHs enriched in the low and high Percoll® (FIG. 7c). This intensity change was not visually seen in the normal donor lot after enrichment in either Percoll® thawing medium. Fluorescent signal was quantified for CD163 expression (FIG. 7d). A higher level of marker expression was measured in the fibrotic lots 2118143 and 2116167 on day 14 in PHHs enriched in low Percoll® (2118143 low: 142.9±85.2 RFUs; 2116167 low: 112.9±34.5 RFUs) versus high Percoll® (2118143 high: 49.8±18.1 RFUs; 2116167 high: 86.1±33.2 RFUs). However, it was not significantly different for any donor. The signal on day 7 was significantly higher in the low Percoll® enriched PHHs (2118143 low: 50.9±12.7 RFUs; 2116167 low: 35.3±10.7 RFUs) compared to the high Percoll® enriched PHHs (2118143 high: 14.6±4.8 RFUs; 2116167 high: 16.5±5.7 RFUs) (Supp. FIG. 1d). No significant differences were determined in the normal lot 16117 on days 7 (low 8.1±3.6 vs high 13.7±7.9 RFUs) and 14 (low 25.6±11.9 vs high 11.9±10.0 RFUs) using either Percoll® thawing medium.

Comparison of phenotype and functionality after being enriched in either a low Percoll® or high Percoll® thawing medium Percoll® density gradients may be used for PHH isolations to use a Percoll® density gradient to purify cells, which may lead to increased cell yield, viability, and performance. Percoll® gradients may be used to separate the different cell types in the liver, including Kupffer cells, stellate cells, or liver sinusoidal endothelial cells based on the different densities of Percoll® after centrifugation. The effect of using a Percoll® gradient to isolate hepatocytes has been examined. However, the outcome of using a Percoll® gradient to enrich cryopreserved diseased PHHs post-thaw has not been as thoroughly assessed. This example was performed using PHHs isolated from diseased and normal donors to compare phenotype and functionality after being enriched in either a low Percoll® or high Percoll® thawing medium.

Patients with T2DM show an increased prevalence of NAFLD. The influence of T2DM on NAFLD and vice versa on disease progression is still unclear. However, the pathogenic mechanism between the two most likely interact. Although the T2DM donor lots tested were not classified as diseased, they may demonstrate how each impacts the other, specifically when examining fibrosis. After the T2DM donor PHHs were enriched in the different thawing medias, notable differences in morphology, functionality, and marker expression were observed as follows. PHHs enriched in high Percoll® appeared to have a more diseased morphology compared to PHHs enriched in low Percoll® for donor lot 2113766. Although these high Percoll® enriched PHHs had greater albumin levels and CYP3A4 activity, fibrotic marker expression of CK18 and TGF-β was elevated when the fluorescent signal was quantitated in this donor. However, in donor lot 2118545, these morphology and functional differences were is reversed. Despite a difference in the albumin level between the low and high Percoll® enriched PHHs, the high Percoll® enriched PHHs had less fibrotic marker expression and lipogenesis in this donor.

Upon examination of the medical history, Donor 2113766 had Coronary Artery Disease (CAD) and a history of hypertension. Studies conducted by Vega et al. (Coronary artery disease as a risk factor for metabolic dysfunction-associated steatotic liver disease and fibrosis. Ann. Hepatol. 2024, 29, 101511) found that patients with liver fibrosis or coronary artery disease (CAD) more frequently had T2DM, making it a “confounding factor”. A group of individuals that have been diagnosed with early-stage liver fibrosis (F1) plus T2DM may have an increased risk of more severe liver disease. These individuals have been designated as “rapid progressors”. Donor 2113766 may have been a “rapid progressor” with clear indicators of fibrosis upon histological examination and PHH characterization but was not suspected of this due to their continued “normal” liver function. Liver enzymes may not be a sensitive marker of NAFLD. Completely normal liver enzymes have been measured from some patients with the most severe form of liver disease. This example observed the range of fibrosis across individuals with normal enzyme values, designating these patients as “normal” yet in reality were “diseased”.

Donor 2113766 had a BMI of 23.9, and donor 2118545 had a BMI of 28.6. Donor 2118545 has a NAS score of 2 with a steatosis score 1, while donor 2113766 has a NAS score of 1 with a steatosis score 0. However, when Nile Red fluorescence was measured, donor lot 2113766 had increased fluorescence compared to donor lot 2118545. In addition to Donor 2113766 being diagnosed with CAD, this donor was Hispanic. It was determined that there is a 31% prevalence of hepatic steatosis in South America. This donor may have been more susceptible to steatosis and increased lipogenesis due to their ethnicity and medical history as well.

In addition to testing T2DM donor lots, diseased lots with NAS scores≥4 were examined. Two of these donor lots, 1811122 and 16096, had NAS scores of 5 and 4, which includes their steatosis score. Both lots showed similar diseased morphology and had no significant differences in attachment after enrichment in either low or high Percoll®. However, when Nile Red fluorescence was quantified, there were significant differences between the low and high Percoll® enriched PHHs. Those PHHs enriched in the low Percoll® had significantly more lipid accumulation compared to the PHHs enriched in the high Percoll®. This occurrence in the diseased PHHs has been seen during hepatocyte isolation from mice with fatty liver. When a low Percoll® gradient (25%) was used during the isolation, there were higher numbers of lipid-filled hepatocytes present compared to the isolation that used a high Percoll® gradient (90%). A similar occurrence, i.e. higher numbers of lipid-filled hepatocytes present in a lower Percoll® gradient compared to an isolation using a higher Percoll® gradient may also happen with the use of higher and lower Percoll® percentages post thaw.

The expression of the gluconeogenesis genes, G6PC and PCK1, was examined in these donors. Both diseased donor lots had significantly lower G6PC gene expression after PHHs were enriched using low Percoll®. Although a similar result was seen for both diseased donor lots for PCK1 gene expression, only donor lot 16096 had significantly lower expression when the PHHs were enriched using low Percoll®. A study by Ye, Q. et al. (Deficiency of gluconeogenic enzyme PCK1 promotes metabolic-associated fatty liver disease through PI3K/AKT/PDGF axis activation in male mice. Nat. Commun. 14, 1402 (2023)) showed that PCK1 is downregulated in NASH patients. This study also found that reduction of PCK1 stimulated inflammation and fibrogenesis in MAFLD mice. The results found in the present gene expression experiments suggest that donor lot 1811122 may have a homogenous population of diseased PHHs that cannot be separated out with the use of different Percoll® densities. However, donor lot 16096 may have a heterogenous population of diseased PHHs that can be distinguished from the other by using specific Percoll® densities. These PHHs (in lot 16096) may be undergoing a progression of disease that allows the diseased hepatocytes to be distinct enough to be separated from other hepatocytes. However, for donor lot 1811122, the majority of PHHs have similar characteristics, where this distinction in population is not as detectable.

Further evidence of this is demonstrated when examining functionality differences after PHHs were enriched in either low or high Percoll®. Only diseased donor lot 16096 showed a significant decrease in albumin and urea levels after enrichment using low Percoll® compared to those PHHs enriched using high Percoll®. There were no significant differences in albumin and urea levels in PHHs after enrichment using either low or high Percoll® for diseased donor lot 1811122. Baseline CYP3A4 activity was significantly higher in the PHHs enriched in the high Percoll® in both diseased lots and the normal lot. Overall, donor lot 1811122 had the lowest urea is level and CYP activity compared to the other two lots. This donor had a hepatocyte ballooning score of 2, which is higher than the score of 1 for donor 16096. It has been found that urea synthesis and CYP3A4 activity but not albumin secretion was reduced in induced human ballooned hepatocytes in a 3D model. It may be due to the increased steatosis and hepatocyte ballooning scores that a homogenous cell population was enriched regardless of the amount of Percoll® in the thawing medium for donor lot 1811122. Donor lot 16096 (diseased) had a heterogenous population of cells that could be separated based on different amounts of Percoll® due to a less severe NAS score, which included a less severe ballooning score. The normal donor lot 16117 has a homogenous population of cells with healthy cells being relatively uniform in their morphology and functionality.

Donor lots 2118143 and 2116167, representing high fibrosis, were the final lots used to determine the outcome on PHHs separation after using either low or high Percoll® post-thaw. Differences in morphology, attachment, lipogenesis, and functionality were seen in PHHs enriched using low Percoll® when compared to the PHHs enriched using high Percoll®. It may be that because both donor lots had a steatosis score of 1, that significantly higher values of Nile Red fluorescence were measured in the low Percoll® enriched PHHs. Increased steatosis may also explain the decline of albumin and urea activity in the low Percoll® enriched PHHs. A decline in serum albumin concentration has been associated with “serious events” in NAFLD patients with steatosis being a defining pathological condition. Studies have also shown urea cycle dysregulation in NASH patients. Mitochondrial urea cycle enzymes were reduced leading to hyperammonia and reduced hepatic capacity to synthesize urea through the urea cycle. Because lipogenesis was lowered in the PHHs enriched in the high Percoll®, there was no decrease in albumin and urea function.

Expression of the fibrosis marker CK18 was significantly higher in the PHHs enriched using the low Percoll® on both days 7 and 14. It may be that the enriched PHHs from these donors could have varying levels of fibrosis that are not determined until the cells are cultured. This suggests that the state of fibrosis could have worsened over time in these donors. No significant differences in CK18 expression were seen in the normal donor lot 16117 after enrichment in either low or high Percoll® on days 7 and 14, further suggesting this donor had a homogenous non-fibrotic population of PHHs.

Notably, only diseased fibrotic donor lot 2116167 (NAS score 4) had significantly higher CD68 expression on day 14 in the low Percoll® enriched PHHs compared to the high Percoll® enriched PHHs. This lot also had the highest fluorescent values. It is interesting to note that fibrotic lot 2118143 (NAS score 3) did not have significant differences in fluorescent values between the two different Percoll® enriched PHHs. The higher level of CD68 marker expression in donor lot 2116167 may be attributable to the additional NAS score of hepatocyte ballooning, which donor lot 2118143 does not have. CD68-positive expressing cells are commonly designated as pro-inflammatory macrophages because they secrete pro-inflammatory cytokines. It has been shown that hepatocyte ballooning is associated with increased inflammation. The presence of hepatocyte ballooning indicates an increased risk of more severe liver disease outcomes and is therefore considered a tipping point in the progression of liver disease. The significantly higher expression of CD68 in the low Percoll® enriched PHHs could mean an increased level of inflammation is occurring in donor lot 2116167 that is not seen in donor lot 2118143.

In addition to CD68 expression, both fibrotic lots expressed CD163. No significant differences were seen between PHHs enriched using low versus high Percoll®. CD163-positive cells are referred to as anti-inflammatory macrophages because they secrete anti-inflammatory cytokines. They have also been shown to be involved in tissue repair which may be pro-fibrotic. These macrophages can express arginase that will then stimulate the synthesis of glutamate and proline, two essential components for collagen synthesis. Therefore, it may be that neither donor lot displays significant differences in CD163 expression levels between PHHs enriched using the low and high Percoll® because of the dual anti-inflammatory/tissue repair functionality of CD163-positive cells.

In conclusion, PHHs were able to be separated into distinct populations using low and high Percoll® after thawing. These distinctions were based on diseased characteristics including steatosis, inflammation, and fibrosis. Variations in functionality and marker expression were also seen from these divergent populations. Overall, the use of differing levels of Percoll® to enrich diseased PHHs allows retention of a diseased phenotype with changes in attachment, function, and lipogenesis. In addition, alterations in fibrotic and inflammatory marker expression can also be determined through the use of different Percoll®-based thawing medias enabling selection of is the best choice for post-thaw processing based on the desired endpoint measures.

All documents, books, manuals, papers, patents, published patent applications, guides, abstracts, and/or other references cited herein are incorporated by reference in their entirety. Other embodiments of the invention will be apparent to those skilled in the art from consideration of the specification and practice of the invention disclosed herein. It is intended that the specification and examples be considered as exemplary only, with the true scope and spirit of the invention being indicated by the following claims.

What is claimed:

1. A method for preparing pooled enriched cells, comprising:
(a) obtaining primary cells from two or more donors;
(b) combining the human primary cells from the two or more donors, whereby combined primary cells are generated;
(c) removing dying or dead cells from the combined human primary cells by a first density gradient separation, whereby combined separated cells are generated;
(d) cryopreserving the combined separated cells, whereby cryopreserved combined separated cells are generated;
(e) thawing the cryopreserved combined separated cells, whereby thawed combined separated cells are generated; and
(f) removing dying or dead cells from the thawed combined separated cells by a second density gradient separation, whereby pooled enriched cells are prepared, wherein the pooled enriched cells have a viability rate greater than that of the primary cells from at least one of the two or more donors.

2. A method for preparing pooled enriched cells, comprising:
(a) obtaining primary cells from two or more donors;
(b) removing dying or dead cells from the primary cells from each of the two or more donors by a first density gradient separation, whereby separated cells from each of the two or more donors are generated;
(c) combining the separated cells from the two or more donors, whereby combined separated cells are generated;
(d) cryopreserving the combined separated cells, cryopreserved combined separated cells are generated;
(e) thawing the cryopreserved combined separated cells, whereby thawed combined separated cells are generated; and
(f) removing dying or dead cells from the thawed combined separated cells by a second density gradient separation, whereby pooled enriched cells are prepared, wherein the pooled enriched cells have a viability rate greater than that of the human primary cells from at least one of the two or more donors;
wherein a total concentration of a density gradient solution in the second density gradient separation is lower than a total concentration of a density gradient solution in the first density gradient separation.

3. A method of preparing pooled enriched cells, comprising:
(a) obtaining primary cells from two or more donors;
(b) removing dying or dead cells from the primary cells from each of the two or more donors by a first density gradient separation whereby density gradient treated cells are generated;
(c) cryopreserving the density gradient treated primary cells to obtain cryopreserved density gradient treated primary cells from each of the two or more donors;
(d) thawing the cryopreserved density gradient treated primary cells from each of the two or more donors whereby thawed density gradient treated primary cells from each of the two or more donors are generated;
(e) combining the primary cells from the two or more donors, whereby combined primary cells are generated;
(f) removing dying or dead cells from the combined human primary cells by a second density gradient separation, whereby combined separated cells are generated;
(g) cryopreserving the combined separated cells, whereby cryopreserved combined separated cells are generated;
(h) thawing the cryopreserved combined separated cells, whereby thawed combined separated cells are generated; and
(i) removing dying or dead cells from the thawed combined separated cells by a third density gradient separation, whereby pooled enriched cells are prepared, wherein the pooled enriched cells have a viability rate greater than that of the human primary cells from at least one of the two or more donors;
wherein a total concentration of a density gradient solution in the third density gradient separation is equal to or higher than a total concentration of a density gradient solution in the second density gradient separation.

4. The method of claim 3, wherein the third density gradient separation is equal to or more stringent than the second density gradient separation.

5. The method of claim 1, wherein the second density gradient separation is equal to or more stringent than the first density gradient separation.

6. The method of claim 1, wherein the pooled enriched cells maintain a viability rate with a less decline than the primary cells from at least one of the two or more donors.

7. The method of claim 1, wherein the pooled enriched cells maintain a biological function with a less decline than the primary cells from at least one of the two or more donors.

8. The method of claim 1, wherein the pooled enriched cells have a lower percentage of cryo-damaged cells than the thawed combined separated cells.

9. A product comprising the pooled enriched cells prepared according to the method of any one of claim 1.

10. A method of testing a pharmaceutical substance, comprising administering a pharmaceutical substance to the pooled enriched cells prepared according to the method of claim 1 in an amount effective to change a property of the pooled enriched cells.

11. A method for preparing enriched cells, comprising:
(a) obtaining primary cells from a donor;
(b) removing dying or dead cells from the primary cells by a first density gradient separation, whereby separated cells are generated;
(c) cryopreserving the separated cells, whereby cryopreserved separated cells are generated;
(d) thawing the cryopreserved separated cells, whereby thawed separated cells are generated; and
(e) removing dying or dead cells from the thawed separated cells by a second density gradient separation, whereby enriched cells are prepared, wherein the enriched cells have a viability rate greater than that of the primary cells from the donor,
wherein a total concentration of a density gradient solution in the second density gradient separation is lower than a total concentration of a density gradient solution in the first density gradient separation.

12. The method of claim 11, wherein the second density gradient separation is more stringent than the first density gradient separation.

13. The method of claim 11, wherein the enriched cells maintain a viability rate with a less decline than the primary cells from the donor.

14. The method of claim 11, wherein the enriched cells maintain a biological function with a less decline than the primary cells from the donor.

15. The method of claim 11, wherein the enriched cells have a lower percentage of cryo-damaged cells than the thawed combined separated cells.

16. A product comprising the enriched cells prepared according to the method of claim 11.

17. A method of testing a pharmaceutical substance, comprising administering a pharmaceutical substance to the enriched cells prepared according to the method of claim 11 in an amount effective to change a property of the enriched cells.
